(19) United States
(12) Patent Application Publication
(10) Pub. No.: US 2026/0182872 A1
(43) Pub. Date: Jul. 2, 2026

(54) SMART NON-INVASIVE HEMOGLOBIN MONITOR

(71) Applicant: Tech4Life Enterprises Canada Inc., Oakville (CA)

(72) Inventors: Shariq Khoja, Oakville (CA); Saad Abdullah, North Karachi (PK); Muhammad Mubashir Ahmed, Karachi (PK); Osama Siddiqui, Karachi (PK)

(73) Assignee: Tech4Life Enterprises Canada Inc., Oakville (CA)

(21) Appl. No.: 19/382,901

(22) Filed: Nov. 7, 2025

Related U.S. Application Data
(60) Provisional application No. 63/719,802, filed on Nov. 13, 2024.

(51) Int. Cl. A61B 5/1455; A61B 5/00

(52) U.S. Cl. CPC A61B 5/1455; A61B 5/6844; A61B 5/742

(57) ABSTRACT

A non-invasive hemoglobin monitoring device comprises processing circuitry coupled to an infrared receiver and a tactile button. The processing circuitry may be configured to detect a finger positioned in a finger placement area based on activation of the tactile button, acquire multiple sensor readings from the infrared receiver as the finger is positioned, calculate an average value from the multiple sensor readings, and determine a hemoglobin concentration based on the average value. A display screen coupled to the processing circuitry may display the hemoglobin concentration. The device may provide accurate hemoglobin measurements without requiring invasive blood sampling procedures, utilizing optical sensing technology and tactile feedback mechanisms to ensure proper finger positioning during measurement operations.

### CROSS REFERENCE TO RELATED APPLICATION

This Application claims the benefit of and priority to U.S. Provisional Application No. 63/719,802, Smart Non-Invasive Hemoglobin Monitor, filed Nov. 13, 2024, which is hereby incorporated by reference in its entirety.

### FIELD

The present disclosure relates to medical diagnostic devices, and more particularly to a non-invasive hemoglobin monitoring device that uses optical sensing technology to measure hemoglobin concentration in blood without requiring blood sampling.

### BACKGROUND

Hemoglobin monitoring plays a fundamental role in medical diagnostics and patient care management. Hemoglobin, the iron-containing protein in red blood cells responsible for oxygen transport throughout the body, serves as a key indicator of various health conditions including anemia, blood loss, and other hematological disorders. Regular monitoring of hemoglobin levels enables healthcare providers to assess patient health status, track treatment progress, and make informed clinical decisions.

Traditional methods for measuring hemoglobin concentration typically involve invasive procedures that require blood sampling through venipuncture or finger pricks. These conventional approaches present several challenges in clinical practice. The invasive nature of blood sampling can cause patient discomfort, anxiety, and pain, particularly problematic for patients requiring frequent monitoring such as those with chronic conditions. Additionally, invasive procedures carry inherent risks including infection, bleeding, and tissue damage at the puncture site.

The invasive nature of conventional hemoglobin testing also creates barriers to widespread screening and routine monitoring. Many patients avoid or delay testing due to discomfort associated with blood draws, potentially leading to delayed diagnosis or inadequate monitoring of existing conditions. This reluctance is particularly pronounced in pediatric populations, elderly patients, and individuals with needle phobias. Furthermore, invasive testing requires trained healthcare personnel, sterile equipment, and proper disposal of biohazardous materials, increasing both cost and complexity of testing procedures.

In clinical settings, the need for frequent hemoglobin monitoring often conflicts with patient comfort and compliance. Patients undergoing treatments such as chemotherapy, dialysis, or surgical procedures may require multiple daily measurements, making invasive testing burdensome for both patients and healthcare staff. The time required for sample collection, processing, and analysis can also delay clinical decision-making, particularly in emergency or critical care situations where rapid assessment is needed.

Non-invasive techniques would address and overcome at least some of the limitations and challenges associated with the traditional invasive techniques.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A smart non-invasive hemoglobin measuring device is disclosed as having processing circuitry coupled to a memory, the processing circuitry to facilitate non-invasive monitoring or measuring of hemoglobin concentration in blood of a user.

The foregoing general description of the illustrative embodiments and the following detailed description thereof are merely exemplary aspects of the teachings of this disclosure and are not restrictive or limiting in any way.

### BRIEF DESCRIPTION OF FIGURES

Non-limiting and non-exhaustive examples are described with reference to the following figures.

FIG. 1 illustrates a flowchart for a non-invasive hemoglobin monitoring method according to one embodiment.

FIG. 2 illustrates a flowchart for a non-invasive hemoglobin monitoring method according to one embodiment.

FIG. 3 illustrates a flowchart for a non-invasive hemoglobin monitoring method according to one embodiment.

FIG. 4 illustrates a flowchart for a hemoglobin concentration validation method according to one embodiment.

FIG. 5 illustrates a transaction sequence representing a non-invasive hemoglobin monitoring method according to one embodiment.

FIG. 6 illustrates a transaction sequence representing a non-invasive hemoglobin monitoring method according to one embodiment.

FIG. 7 illustrates a transaction sequence representing a non-invasive hemoglobin monitoring method according to one embodiment.

FIG. 8 illustrates a transaction sequence representing a non-invasive hemoglobin monitoring method according to one embodiment.

FIG. 9 illustrates a block diagram of a non-invasive hemoglobin monitoring device housing according to one embodiment.

FIG. 10 illustrates a signal processing architecture of an embodiment of the novel smart non-invasive hemoglobin monitoring device according to one embodiment.

FIG. 11 illustrates an exploded view of a signal processing architecture of an embodiment of the novel smart non-invasive hemoglobin monitoring device according to one embodiment.

FIG. 12 illustrates various views depicting an assembly sequence of a device housing of an embodiment of the novel smart non-invasive hemoglobin monitoring device according to one embodiment.

FIG. 13 illustrates an exploded view of a device architecture of an embodiment of the novel smart non-invasive hemoglobin monitoring device according to one embodiment.

FIG. 14 illustrates a diagrammatic representation of a machine in the exemplary form of a computer system capable of implementing embodiments disclosed in this document.

FIG. 15 illustrates a block diagram of a power management system of an embodiment of the novel smart non-invasive hemoglobin monitoring device according to one embodiment.

FIG. 16 illustrates a device communication architecture of an embodiment of the novel smart non-invasive hemoglobin monitoring device according to one embodiment.

FIG. 17 illustrates a transmitter sensor connector circuit schematic for the novel smart non-invasive hemoglobin monitoring device according to one embodiment.

FIG. 18 depicts a receiver sensor connector circuit schematic for the novel smart non-invasive hemoglobin monitoring device according to one embodiment.

FIG. 19 illustrates a finger detection circuit system of an embodiment of the novel smart non-invasive hemoglobin monitoring device according to one embodiment.

FIG. 20 illustrates a battery power management system of an embodiment of the novel smart non-invasive hemoglobin monitoring device according to one embodiment.

FIG. 21 illustrates a 5 volts booster circuit system of an embodiment of the novel smart non-invasive hemoglobin monitoring device according to one embodiment.

FIG. 22 illustrates a switching circuit system of an embodiment of the novel smart non-invasive hemoglobin monitoring device according to one embodiment.

FIG. 23 illustrates a switching circuit component schematic of an embodiment of the novel smart non-invasive hemoglobin monitoring device according to one embodiment.

FIG. 24 illustrates an optical sensing system of an embodiment of the novel smart non-invasive hemoglobin monitoring device according to one embodiment.

FIG. 25 illustrates a charging protection circuit system of an embodiment of the novel smart non-invasive hemoglobin monitoring device according to one embodiment.

FIG. 26 illustrates a programming interface system of an embodiment of the novel smart non-invasive hemoglobin monitoring device according to one embodiment.

FIG. 27 illustrates a microcontroller programming interface circuit of an embodiment of the novel smart non-invasive hemoglobin monitoring device according to one embodiment.

FIG. 28 illustrates a transmitter sensor connector circuit of an embodiment of the novel smart non-invasive hemoglobin monitoring device according to one embodiment.

FIG. 29 illustrates a device calibration system of an embodiment of the novel smart non-invasive hemoglobin monitoring device according to one embodiment.

FIG. 30 illustrates a microcontroller unit circuit system of an embodiment of the novel smart non-invasive hemoglobin monitoring device according to one embodiment.

FIG. 31 illustrates a Thin-Film Transistor display interface system of an embodiment of the novel smart non-invasive hemoglobin monitoring device according to one embodiment.

FIG. 32 illustrates a battery monitoring circuit system of an embodiment of the novel smart non-invasive hemoglobin monitoring device according to one embodiment.

FIG. 33 illustrates a finger detection circuit with light-emitting diode components of an embodiment of the novel smart non-invasive hemoglobin monitoring device according to one embodiment.

FIG. 34 illustrates a block diagram of an optical measurement system of an embodiment of the novel smart non-invasive hemoglobin monitoring device according to one embodiment.

FIG. 35 illustrates a device housing assembly of an embodiment of the novel smart non-invasive hemoglobin monitoring device according to one embodiment.

FIG. 36 illustrates a block diagram of a device enclosure system of an embodiment of the novel smart non-invasive hemoglobin monitoring device according to one embodiment.

FIG. 37 illustrates a sequence representing a device initialization system of an embodiment of the novel smart non-invasive hemoglobin monitoring device according to one embodiment.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

Non-invasive measurement technologies have emerged as an alternative approach to address these limitations. These technologies typically employ optical methods that analyze light absorption or reflection characteristics of blood components through the skin. However, existing non-invasive hemoglobin measurement devices face challenges related to measurement accuracy, consistency, and reliability across different patient populations and environmental conditions. Factors such as skin pigmentation, tissue thickness, ambient light, and patient movement can affect measurement quality and introduce variability in results.

Portable and user-friendly medical devices have become increasingly important in modern healthcare delivery, particularly for point-of-care testing and home monitoring applications. The ability to perform reliable measurements outside traditional laboratory settings enables more flexible patient care, reduces healthcare costs, and improves patient access to diagnostic testing. However, developing portable devices that maintain clinical accuracy while providing ease of use remains a technical challenge in the field of medical diagnostics.

According to an aspect of the present disclosure, a non-invasive hemoglobin monitoring device (herein referred to as “smart device”, “smart monitoring device”, “monitoring device”, or simply “device”) is provided. The device comprises a housing having a finger placement area. The device comprises an infrared transmitter positioned within the housing and configured to emit light at a wavelength, such as 880 nanometers (“nm”), etc., through the finger placement area, where this wavelength may include but is not limited to 880 nm. It is contemplated and understood that any particular wavelength, such as 880 nm, 940 nm, etc., have been referenced merely as examples throughout this document and that this novel non-invasive hemoglobin device is capable of adapting to any wavelength and is in no way limited to any particular wavelength or any examples of wavelengths recited in this document. The device comprises an infrared receiver positioned within the housing and configured to detect the light transmitted through a finger placed in the finger placement area. The device comprises a tactile button positioned within the finger placement area and configured to be pressed when a finger is properly positioned. The device comprises processing circuitry coupled to the infrared receiver and the tactile button, the processing circuitry configured to determine when the finger is properly positioned based on activation of the tactile button, acquire multiple sensor readings from the infrared receiver when the finger is properly positioned, calculate an average value from the multiple sensor readings, and determine a hemoglobin concentration based on the average value using a predetermined algorithm. The device comprises a display coupled to the processing circuitry and configured to display the determined hemoglobin concentration.

According to other aspects of the present disclosure, the device may include one or more of the following features. The processing circuitry may be configured to acquire 20 sensor readings from the infrared receiver with a 0.5-second delay between each reading. The processing circuitry may be further configured to validate the determined hemoglobin concentration by checking whether the concentration falls within a predetermined range of 1 to 17.5 grams per deciliter. The processing circuitry may be configured to display an error message on the display when the determined hemoglobin concentration falls outside the predetermined range. The predetermined algorithm may comprise selecting between a first calculation formula and a second calculation formula based on whether the average value is less than 1017 or greater than or equal to 1017. The housing may comprise a top enclosure and a bottom enclosure connected by a vertical opening mechanism. The top enclosure may house the infrared transmitter and the display, and the bottom enclosure may house a battery and the infrared receiver.

According to another aspect of the present disclosure, a method for non-invasively monitoring hemoglobin concentration is provided. The method comprises emitting infrared light at a wavelength, such as 880 nanometers, from a transmitter through a finger placement area of a monitoring device. The method comprises detecting the infrared light with a receiver after the light has passed through a finger positioned in the finger placement area. The method comprises determining proper finger positioning based on activation of a tactile button positioned within the finger placement area. The method comprises acquiring multiple sensor readings from the receiver when proper finger positioning is confirmed. The method comprises calculating an average value from the multiple sensor readings. The method comprises determining a hemoglobin concentration based on the average value using a predetermined algorithm. The method comprises displaying the determined hemoglobin concentration on a display of the monitoring device.

According to other aspects of the present disclosure, the method may include one or more of the following features. Acquiring multiple sensor readings may comprise acquiring 20 sensor readings from the receiver with a 0.5-second delay between each reading. The method may further comprise a step of validating the determined hemoglobin concentration by checking whether the concentration falls within a predetermined range of 1 to 17.5 grams per deciliter. The method may further comprise a step of displaying an error message on the display when the determined hemoglobin concentration falls outside the predetermined range. Determining the hemoglobin concentration may comprise selecting between a first calculation formula and a second calculation formula based on whether the average value is less than 1017 or greater than or equal to 1017. Calculating the average value may comprise averaging the 20 sensor readings to produce a single representative value for hemoglobin calculation. The error message may indicate improper finger placement and prompts repositioning of the finger in the finger placement area.

According to another aspect of the present disclosure, a portable hemoglobin measurement system is provided. The system comprises a vertically opening housing configured to receive a finger. The system comprises optical sensing components including an infrared light source operating at a given wavelength, such as 880 nanometers, and a photodetector positioned to measure light transmission through the finger. The system comprises a finger positioning mechanism including a tactile button that must be activated to confirm proper finger placement. The system comprises signal processing electronics configured to convert analog signals from the photodetector to digital values and calculate hemoglobin concentration using a compartmental model algorithm. The system comprises a rechargeable battery power supply. The system comprises a digital display for presenting measurement results.

According to other aspects of the present disclosure, the system may include one or more of the following features. The signal processing electronics may comprise a microcontroller configured to acquire 20 sensor readings from the photodetector with a 0.5-second delay between each reading. The microcontroller may be further configured to calculate an average value from the 20 sensor readings and apply the compartmental model algorithm based on whether the average value is less than 1017 or greater than or equal to 1017. The vertically opening housing may comprise a top enclosure housing the infrared light source and the digital display, and a bottom enclosure housing the rechargeable battery and the photo detector. The signal processing electronics may be further configured to validate a calculated hemoglobin concentration by determining whether the concentration falls within a range of 1 to 17.5 grams per deciliter. The digital display may be configured to present an error message when the calculated hemoglobin concentration falls outside the range of 1 to 17.5 grams per deciliter.

Non-invasive hemoglobin monitoring represents a technological advancement in medical diagnostics that eliminates the discomfort and infection risks associated with traditional blood sampling methods. Such monitoring devices utilize optical sensing technology to assess hemoglobin concentration through tissue analysis, providing healthcare professionals and patients with a convenient alternative to conventional invasive testing procedures. The development of portable, user-friendly hemoglobin monitoring systems addresses the growing demand for accessible diagnostic tools that can be deployed in various healthcare settings, from clinical environments to home-based monitoring applications.

Certain modern hemoglobin monitoring techniques incorporate sophisticated signal processing algorithms and optical components to achieve accurate measurements through non-invasive means. These systems typically employ infrared light transmission techniques that analyze the absorption characteristics of hemoglobin in blood vessels beneath the skin surface. The integration of digital processing capabilities with optical sensing technology enables real-time measurement and display of hemoglobin concentration values, facilitating immediate clinical decision-making and patient assessment. Such devices may be particularly beneficial for individuals requiring frequent hemoglobin monitoring, including patients with anemia, chronic kidney disease, or other conditions affecting blood composition.

The portability and ease of use of contemporary hemoglobin monitoring systems make them suitable for deployment across diverse healthcare scenarios. Healthcare providers may utilize these devices in outpatient clinics, emergency departments, and remote monitoring programs where rapid hemoglobin assessment is valuable. The non-invasive nature of the measurement process encourages patient compliance and reduces the procedural burden associated with repeated blood draws. Additionally, the compact form factor of modern devices enables their use in resource-limited settings where traditional laboratory-based hemoglobin testing may not be readily available.

Technological advancements in optical sensing, signal processing, and user interface design have contributed to the development of hemoglobin monitoring devices that combine measurement accuracy with operational simplicity. These systems incorporate various electronic components, including microcontrollers, analog-to-digital converters, and display modules, to provide comprehensive measurement and data presentation capabilities. The integration of power management systems and rechargeable battery technology enables extended operational periods, supporting continuous monitoring applications and reducing the frequency of device maintenance requirements.

The housing structure of non-invasive hemoglobin monitoring devices incorporates a specialized design that facilitates optical measurement while ensuring user comfort and device portability. The housing may include a finger placement area specifically configured to accommodate a user's finger during measurement procedures. This finger placement area may be dimensioned to provide proper alignment between the user's finger and the internal optical components, enabling consistent light transmission paths for accurate hemoglobin concentration determination. The finger placement area may incorporate ergonomic features that guide finger positioning and maintain stable contact throughout the measurement process.

The housing may comprise a top enclosure and a bottom enclosure connected through a vertical opening mechanism that enables access to internal components while maintaining structural integrity during operation. The vertical opening mechanism may allow the housing to separate along a predetermined axis, providing convenient access for component maintenance, battery replacement, or internal cleaning procedures. In some cases, the top enclosure houses the infrared transmitter and display components, while the bottom enclosure accommodates the battery and infrared receiver elements. This configuration may optimize the optical path between transmitter and receiver components while distributing weight evenly across the device structure.

The construction materials for the housing may include medical-grade plastics such as polycarbonate or acrylonitrile butadiene styrene (ABS) that provide durability, biocompatibility, and resistance to cleaning agents commonly used in healthcare environments. Polycarbonate materials may offer transparency properties that facilitate visual inspection of internal components, while ABS materials may provide enhanced impact resistance and dimensional stability under varying temperature conditions. The selection of housing materials may consider factors including sterilization compatibility, chemical resistance, and long-term stability under repeated use conditions.

The housing design may incorporate internal mounting structures that secure electronic components in predetermined positions relative to the finger placement area and optical sensing elements. These mounting structures may include ribs, posts, or recessed areas that prevent component movement during device operation and transportation. The internal layout may be configured to minimize electromagnetic interference between electronic components while maintaining compact overall dimensions. In some cases, the housing may include cable management features that organize internal wiring and prevent interference with moving parts associated with the vertical opening mechanism.

Light indication systems within the housing may utilize light pipe structures that transfer optical signals from internal light-emitting components to visible locations on the exterior housing surface. The light pipe may comprise transparent or translucent materials that conduct light through internal reflection, enabling status indicators to be visible from the front side of the device body without requiring direct mounting of light sources on the exterior surface. This light pipe configuration may provide better visibility of device status indicators while protecting internal light sources from environmental exposure and physical damage. The light pipe may be integrated into the housing structure during manufacturing processes or installed as separate components within designated channels or cavities.

Embodiments may be implemented as any or a combination of one or more microchips or integrated circuits interconnected using a parentboard, hardwired logic, software stored by a memory device and executed by a microprocessor, firmware, an application specific integrated circuit (ASIC), and/or a field programmable gate array (FPGA). Terms like “logic”, “module”, “component”, “engine”, “circuitry”, “element”, and “mechanism” may include, by way of example, software, hardware, firmware, and/or any combination thereof.

In this description, numerous specific details are set forth. However, embodiments, as described herein, may be practiced without these specific details. In other instances, well-known circuits, structures and techniques have not been shown in detail in order not to obscure the understanding of this description. It is contemplated that embodiments are not limited to any number or types of processes, materials, apparatus, or techniques for achieving the novel soldering techniques in electronics and semiconductor manufacturing environments.

Throughout this document, terms like “logic”, “component”, “module”, “framework”, “engine”, “mechanism”, “technique”, and/or the like, may be referenced interchangeably and include, by way of example, software, hardware, and/or any combination of software and hardware, such as firmware. Further, any use of a particular brand, word, term, phrase, name, acronym, or the like, such as “device”, “smart device”, “smart hemoglobin monitor”, “smart non-invasive hemoglobin monitor device”, “SmartHb”, “Smart-Hb”, “Smart-HB hemoglobin device”, “non-invasive”, “smart”, “circuits”, “electronics”, “semiconductor”, “user”, “patient”, “material”, “wireless”, “computing device”, “smartphone”, “tablet computer”, “software application”, “social and/or business networking applications or websites”, “website”, or “site”, and/or the like, should not be read to limit embodiments to software or devices that carry that label in products or in literature external to this document.

Smart non-invasive hemoglobin monitoring device may host network interface device(s) to provide access to a network, such as a LAN, a wide area network (WAN), a metropolitan area network (MAN), a personal area network (PAN), Bluetooth, a cloud network, a mobile network (e.g., 3rd Generation (3G), 4th Generation (4G), 5th Generation (5G), etc.), an intranet, the Internet, etc. Network interface(s) may include, for example, a wireless network interface having antenna, which may represent one or more antenna(e). Network interface(s) may also include, for example, a wired network interface to communicate with remote devices via network cable, which may be, for example, an Ethernet cable, a coaxial cable, a fiber optic cable, a serial cable, or a parallel cable.

Smart non-invasive hemoglobin monitoring device may host network interface device(s) to provide access to a network, such as a LAN, a wide area network (WAN), a metropolitan area network (MAN), a personal area network (PAN), Bluetooth, a cloud network, a mobile network (e.g., 3rd Generation (3G), 4th Generation (4G), 5th Generation (5G), etc.), an intranet, the Internet, etc. Network interface(s) may include, for example, a wireless network interface having antenna, which may represent one or more antenna(e). Network interface(s) may also include, for example, a wired network interface to communicate with remote devices via network cable, which may be, for example, an Ethernet cable, a coaxial cable, a fiber optic cable, a serial cable, or a parallel cable.

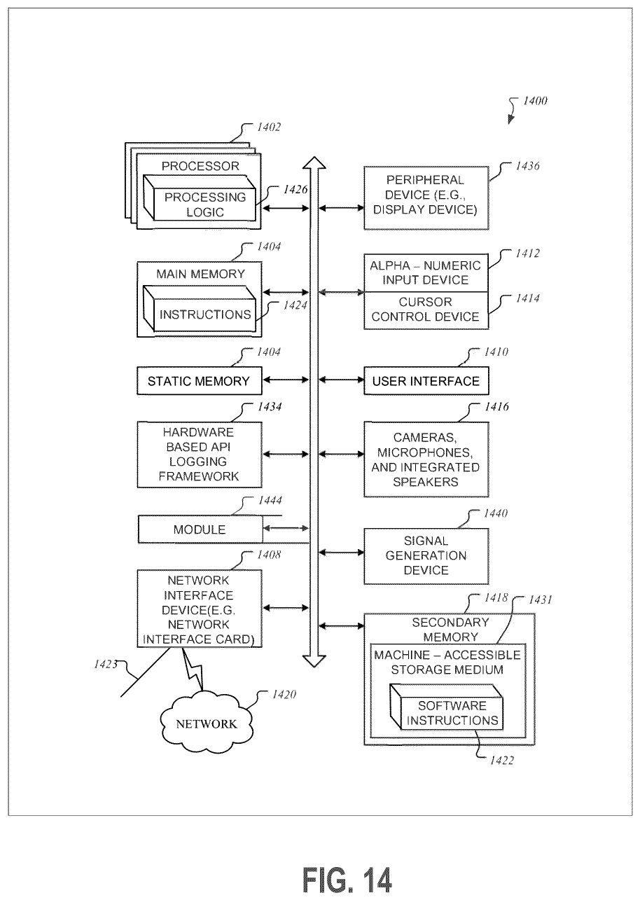

Embodiments may be provided, for example, as a computer program product which may include one or more machine-readable media having stored thereon machine-executable instructions that, when executed by one or more machines such as a computer, a data processing machine, a data processing device, network of computers, or other electronic devices, may result in the one or more machines carrying out operations in accordance with embodiments described herein. As further described with reference to processing architecture 1400 of FIG. 14, a machine may include one or more processors, such as a CPU, a GPU, etc. A machine-readable medium may include, but is not limited to, floppy diskettes, optical disks, Compact Disc-Read Only Memories (CD-ROMs), magneto-optical disks, ROMs, Random Access Memories (RAMs), Erasable Programmable Read Only Memories (EPROMs), Electrically Erasable Programmable Read Only Memories (EEPROMs), magnetic or optical cards, flash memory, or other type of media/machine-readable medium suitable for storing machine-executable instructions.

For example, when reading any of the apparatus, method, or system claims of this patent to cover a purely software and/or firmware implementation, instructions associated with hemoglobin monitoring mechanism may be expressly stored at a non-transitory computer readable storage device or storage disk such as a memory, a digital versatile disk (DVD), a compact disk (CD), a Blu-ray disk, etc., including the software and/or firmware.

Moreover, one or more elements of hemoglobin monitoring mechanism may be downloaded as a computer program product, wherein the program may be transferred from a remote computer (e.g., a server) to a requesting computer (e.g., a client) by way of one or more data signals embodied in and/or modulated by a carrier wave or other propagation medium via a communication link (e.g., a modem and/or network connection).

Throughout this document, the term “user” may be interchangeably referred to as “viewer”, “observer”, “speaker”, “person”, “individual”, “end-user”, “developer”, “programmer”, “administrators”, and/or the like. For example, in some cases, a user may refer to an end-user, such as a consumer accessing a client computing device, while, in some other cases, a user may include a developer, a programmer, a system administrator, etc., accessing a workstation serving as a client computing device. It is to be noted that throughout this document, terms like “graphics domain” may be referenced interchangeably with “graphics processing unit”, “graphics processor”, or simply “GPU”; similarly, “CPU domain” or “host domain” may be referenced interchangeably with “computer processing unit”, “application processor”, or simply “CPU”.

It is to be noted that terms like “node”, “computing node”, “server”, “server device”, “cloud computer”, “cloud server”, “cloud server computer”, “machine”, “host machine”, “device”, “computing device”, “computer”, “computing system”, and the like, may be used interchangeably throughout this document. It is to be further noted that terms like “application”, “software application”, “program”, “software program”, “package”, “software package”, and the like, may be used interchangeably throughout this document.

Further, throughout this document, terms like “request”, “query”, “job”, “work”, “work item”, and “workload” are referenced interchangeably. Similarly, an “application” or “agent” may refer to or include a computer program, a software application, a game, a workstation application, etc., offered through an application programming interface (API), such as a free rendering API, such as Open Graphics Library (OpenGL®), DirectX® 11, DirectX® 12, etc., where “dispatch” may be interchangeably referenced as “work unit” or “draw”, while “application” may be interchangeably referred to as “workflow” or simply “agent”.

In some embodiments, terms like “display screen” and “display surface” may be used interchangeably referring to the visible portion of a display device while the rest of the display device may be embedded into a computing device, such as a smartphone, a wearable device, etc. It is contemplated and to be noted that embodiments are not limited to any particular computing device, software application, hardware component, display device, display screen or surface, protocol, standard, etc. For example, embodiments may be applied to and used with any number and type of real-time applications on any number and type of computers, such as desktops, laptops, tablet computers, smartphones, head-mounted displays and other wearable devices, and/or the like. Further, for example, rendering scenarios for efficient performance using this novel technique may range from simple scenarios, such as desktop compositing, to complex scenarios, such as three-dimensional (3D) games, augmented reality applications, etc.

Any of the above embodiments may be used alone or together with one another in any combination. Embodiments encompassed within this specification may also include embodiments that are only partially mentioned or alluded to or are not mentioned or alluded to at all in this brief summary or in the abstract. Although various embodiments may have been motivated by various deficiencies with the prior art, which may be discussed or alluded to in one or more places in the specification, the embodiments do not necessarily address any of these deficiencies. In other words, different embodiments may address different deficiencies that may be discussed in the specification. Some embodiments may only partially address some deficiencies or just one deficiency that may be discussed in the specification, and some embodiments may not address any of these deficiencies.

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements. It is to be understood that the above description is intended to be illustrative, and not restrictive.

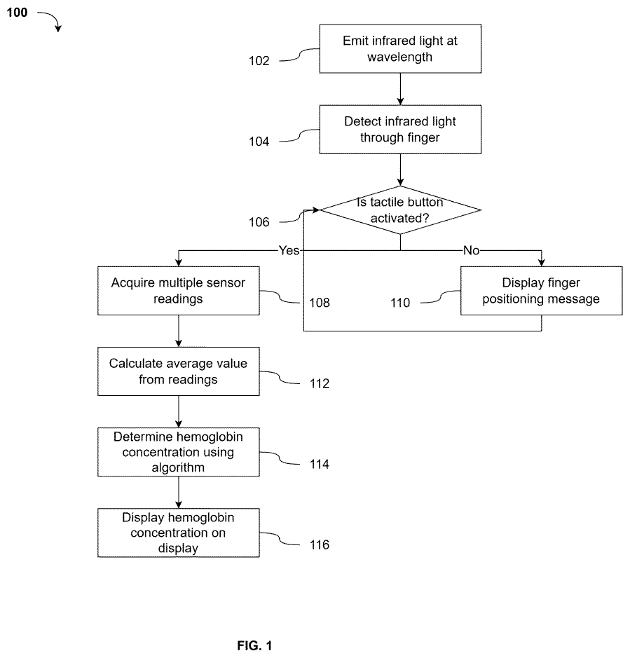

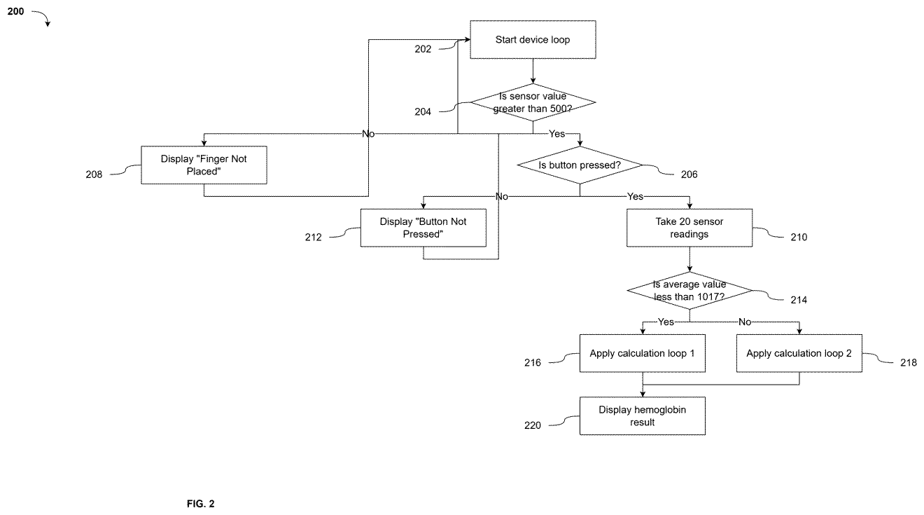

FIG. 1 illustrates a flowchart for a non-invasive hemoglobin monitoring method 100 according to one embodiment. The method 100 begins at block 102, where infrared light is emitted at a certain wavelength, such as 880 nm wavelength. As aforementioned, although 880 nm wavelength is used as an example throughout this document, it is contemplated and should be understood that embodiments are not limited to any particular wavelength. The process then proceeds to block 104, where the infrared light is detected after passing through a finger. The method 100 continues to decision block 106, which presents a decision point determining whether a tactile button is activated. If the tactile button is activated (Yes branch), the method 100 advances to block 108, where multiple sensor readings are acquired. If the tactile button is not activated (No branch), the method 100 proceeds to block 110, where a finger positioning message is displayed, and the process returns to the decision point at decision block 106. Following the acquisition of sensor readings at 108, the method 100 moves to block 112, where an average value is calculated from the readings. The process then continues to block 114, where hemoglobin concentration is determined using an algorithm. Finally, the method 100 concludes at block 116, where the hemoglobin concentration is displayed on a display. The flowchart demonstrates the sequential flow of the non-invasive measurement process, incorporating proper finger positioning verification through the tactile button mechanism and systematic data processing to achieve accurate hemoglobin concentration determination.

FIG. 2 illustrates a flowchart for a non-invasive hemoglobin monitoring method 200 according to one embodiment. The method 200 begins at 202, where the device loop is started. The process then moves to decision block 204, which determines whether the sensor value is greater than 500. It is contemplated that the sensor value of 500 is merely used here and throughout the document as an example and that the embodiments are not limited to any particular sensor value. If the sensor value is not greater than 500 (No branch), the method 200 proceeds to block 208, where “Finger Not Placed” is displayed, and the process returns to block 202 to restart the device loop. If the sensor value is greater than 500 (Yes branch), the method 200 continues to decision block 206, which checks whether the button is pressed. From decision block 206, if the button is not pressed (No branch), the method 200 moves to block 212, where “Button Not Pressed” is displayed, and the process returns to 202. If the button is pressed (Yes branch), the method 200 proceeds to block 210, where a number of sensor readings, such as 20 sensor readings, are taken. Following block 210, the process advances to decision decision 214, which determines whether the average value is less than, for example, 1017. Any values, such as sensor readings of 20 or 1017 are merely examples and that embodiments are not limited to any particular readings or values.

At decision block 214, if the average value is less than, for example, 1017 (Yes branch), the method 200 proceeds to block 216, where calculation loop 1 is applied. If the average value is not less than 1017 (No branch), the method 200 moves to block 218, where calculation loop 2 is applied. Both block 216 and block 218 converge at step block 220, where the hemoglobin result is displayed. The flowchart demonstrates the sequential decision-making process and validation steps required for accurate hemoglobin measurement, incorporating finger placement detection and sensor value processing to ensure reliable measurement results.

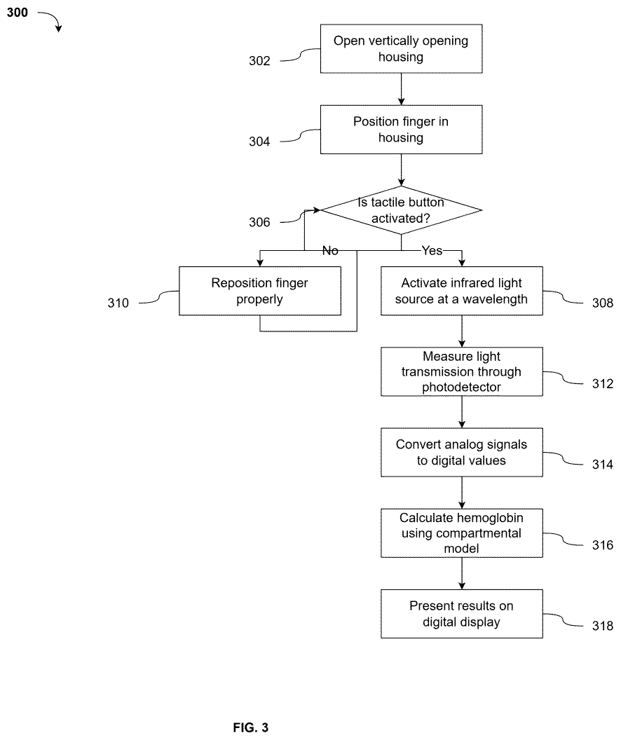

FIG. 3 illustrates a flowchart for a non-invasive hemoglobin monitoring method 300 according to one embodiment. The method 300 begins with block 302, where a vertically opening housing is opened. The process then proceeds to block 304, where a finger is positioned in the housing. The method 300 continues to block 306, which involves a decision point to determine if a tactile button is activated. If the tactile button is not activated (No branch), the method 300 moves to block 310, where the finger is repositioned properly. From block 310, the process returns to block 306 to check again if the tactile button is activated. If the tactile button is activated (Yes branch), the method 300 proceeds to block 308, where an infrared light source is activated at a given wavelength, such as 880 nm. The process then advances to block 312, where light transmission is measured through a photo detector. Following this measurement, the method 300 moves to block 314, where analog signals are converted to digital values. The process continues to block 316, where hemoglobin is calculated using a compartmental model. Finally, the method 300 concludes with block 318, where results are presented on a digital display. The flowchart demonstrates the sequential flow of operations for the portable hemoglobin measurement system, incorporating proper finger positioning verification through the tactile button mechanism before proceeding with optical measurements and signal processing calculations.

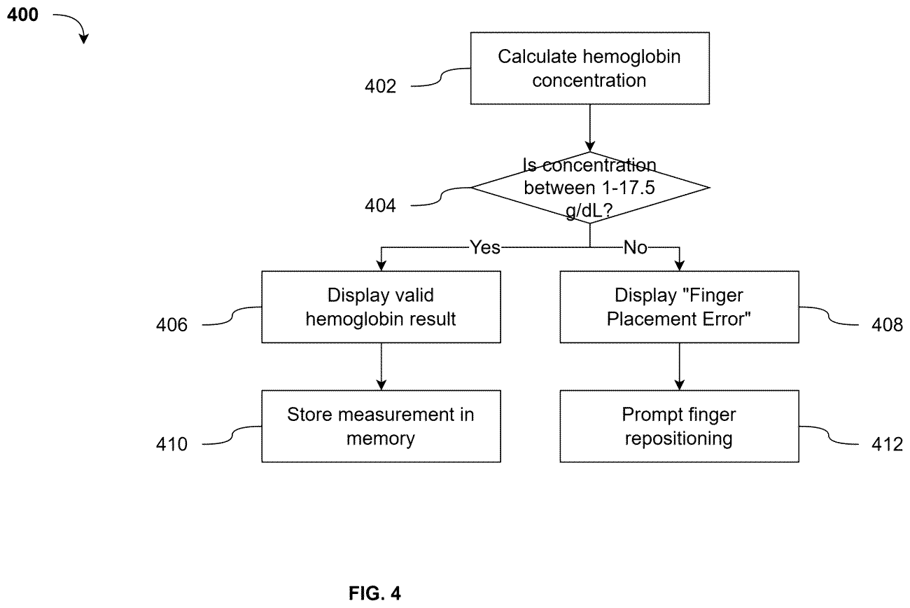

FIG. 4 illustrates a flowchart for a hemoglobin concentration validation method 400 according to one embodiment. The method 400 begins with a block 402, where the hemoglobin concentration is calculated. The method 400 then proceeds to a decision block 404, where the system determines whether the calculated concentration falls, for example, between 1-17.5 g/dL. As discussed, embodiments are not limited or confined to any particular values, measurements, etc., and any numbers, such as calculated concentration falling between 1-17.5 g/dL, is merely used as an example. If the concentration is within the valid range (Yes branch), the method 400 moves to a block 406, where the valid hemoglobin result is displayed to the user. Following the display of valid results, the method 400 continues to a block 410, where the measurement is stored in memory for future reference.

If the concentration falls outside the acceptable range (No branch from decision block 404), the method 400 proceeds to a block 408, where a “Finger Placement Error” message is displayed. After displaying the error message, the method 400 moves to a block 412, where the system prompts the user for finger repositioning to obtain a more accurate measurement. The flowchart for the method 400 demonstrates the validation process that ensures measurement reliability by checking calculated hemoglobin values against predetermined physiological limits. The method 400 incorporates error handling mechanisms that guide users toward proper device operation when measurements fall outside expected parameters. The method 400 provides a systematic approach to quality control in non-invasive hemoglobin monitoring by distinguishing between valid measurements that should be stored and displayed versus invalid measurements that require user intervention and repositioning.

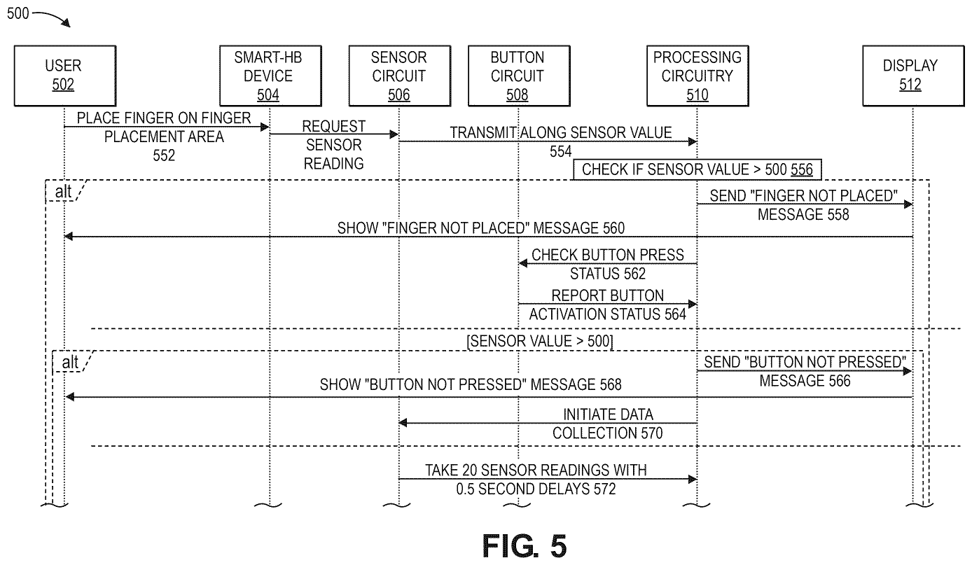

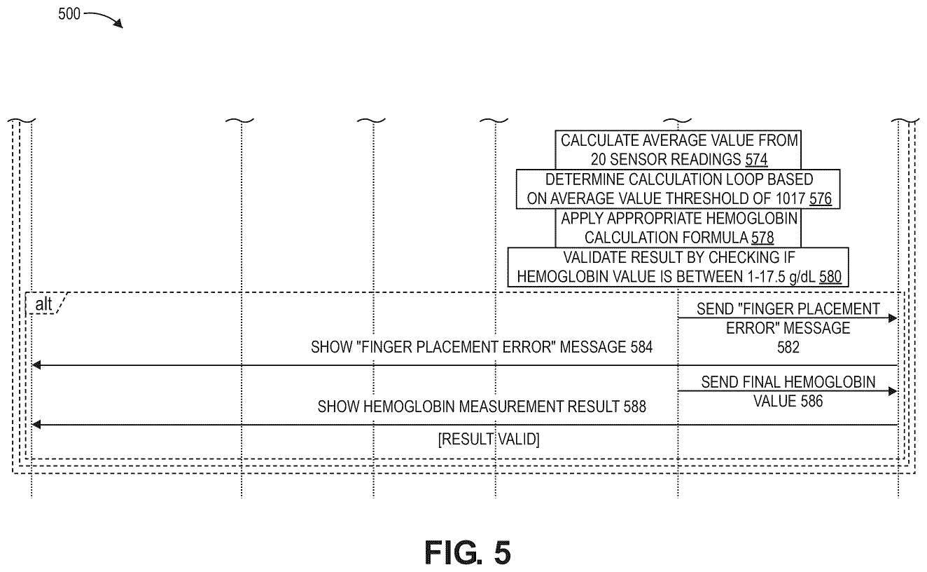

FIG. 5 illustrates a transaction sequence 500 representing a non-invasive hemoglobin monitoring method according to one embodiment. The diagram includes several elements, such as a user 502, a smart non-invasive hemoglobin monitoring device (smart-Hb device) 504, a sensor circuit 506, a button circuit 508, processing circuitry 510, and a display 512. The sequence 500 begins when a user places their finger on a finger placement area at 552. The smart-Hb device 504 sends a request for sensor reading to the sensor circuit 506. The sensor circuit 506 transmits an analog sensor value 554 to the processing circuitry 510. The processing circuitry 510 checks if the sensor value is greater than 500 at 556, and sends a “Finger Not Placed” message 558 if the value is below threshold. The button circuit 508 checks button press status at 562 and reports button activation status 564 to the processing circuitry 510. When the sensor value exceeds 500, the processing circuitry 510 sends a “Button Not Pressed” message 566 to the display 512.

The processing circuitry 510 then initiates data collection at 570 and takes 20 sensor readings with 0.5-second delays at 572. The processing circuitry 510 calculates an average value from the 20 sensor readings in step 574, determines calculation loop based on average value threshold of 1017 at 576, and applies appropriate hemoglobin calculation formula at 578. The processing circuitry 510 validates the result by checking if the hemoglobin value is between 1-17.5 g/dL in at 580. If validation fails, a “Finger Placement Error” message 582 is sent to the display 512. Upon successful validation, the final hemoglobin value 586 is sent to the display 512, which shows the hemoglobin measurement result at 588.

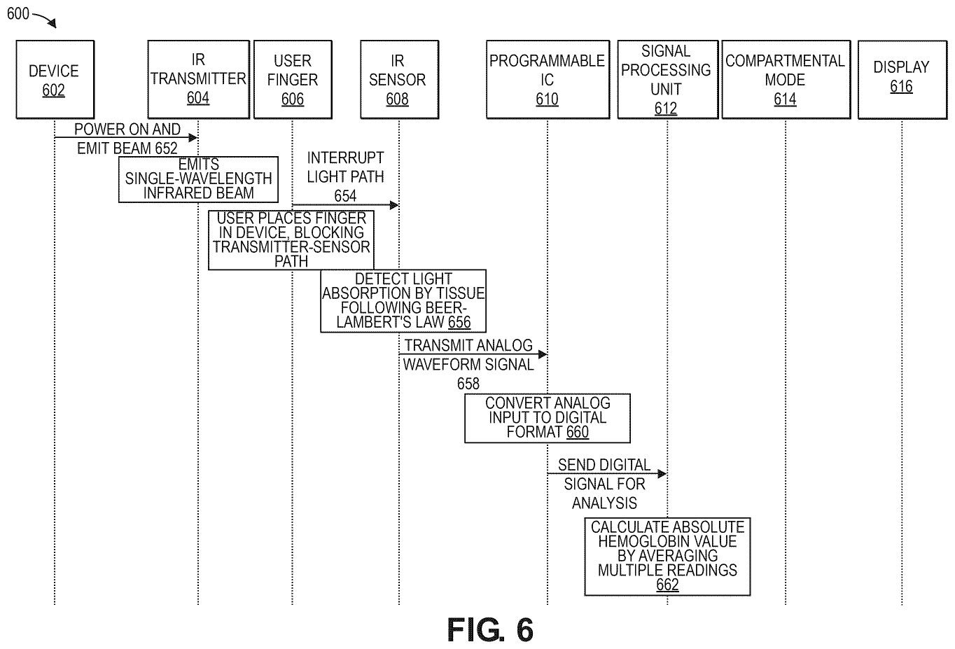

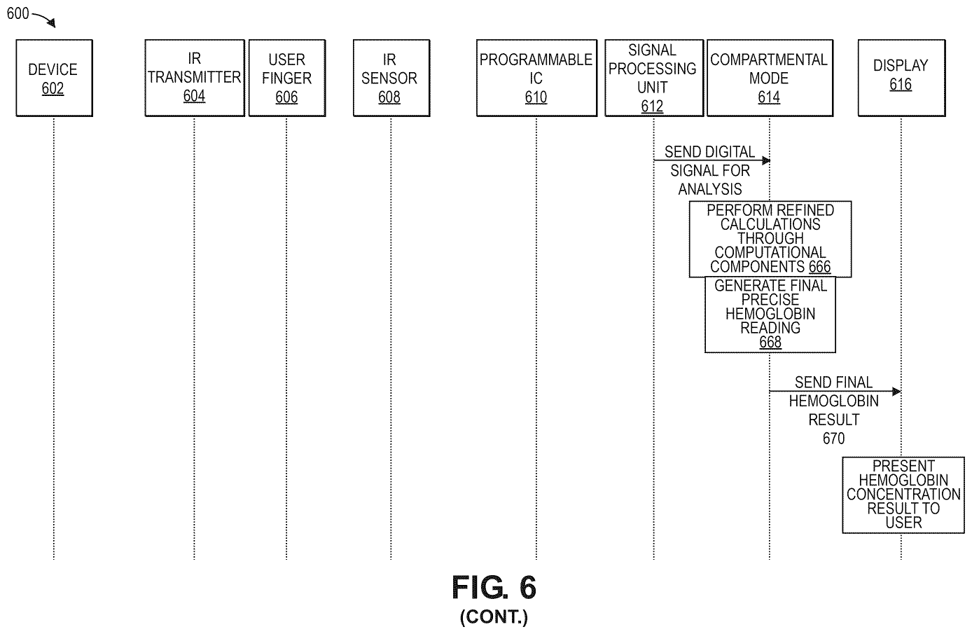

FIG. 6 illustrates a transaction sequence 600 representing a non-invasive hemoglobin monitoring method according to one embodiment. The diagram includes several elements, including a device 602, an infrared transmitter 604, a user finger 606, an infrared sensor 608, a programmable integrated circuit 610, and a signal processing unit 612. The process begins with the device 602 sending a power on and emit (e.g., 880 nm) beam signal 652 to the infrared transmitter 604. The infrared transmitter 604 emits a single-wavelength infrared beam, such as at 880 nm. When a user finger 606 interrupts the light path 654, the user places their finger in the device, blocking the transmitter-sensor path. The infrared sensor 608 detects light absorption by tissue following Beer-Lambert's Law 656. The infrared sensor 608 then transmits an analog waveform signal 658 to the programmable integrated circuit 610.

The programmable integrated circuit 610 converts the analog input to digital format 660 and sends the digital signal for analysis to the signal processing unit 612. The signal processing unit 612 calculates absolute hemoglobin value by averaging multiple readings 662 and sends the absolute value 664 to the compartmental model 614. The compartmental model 614 performs refined calculations through computational compartments 666 and generates a final precise hemoglobin reading 668. The final hemoglobin result 670 is then sent to the display 616, which presents the hemoglobin concentration result to the user.

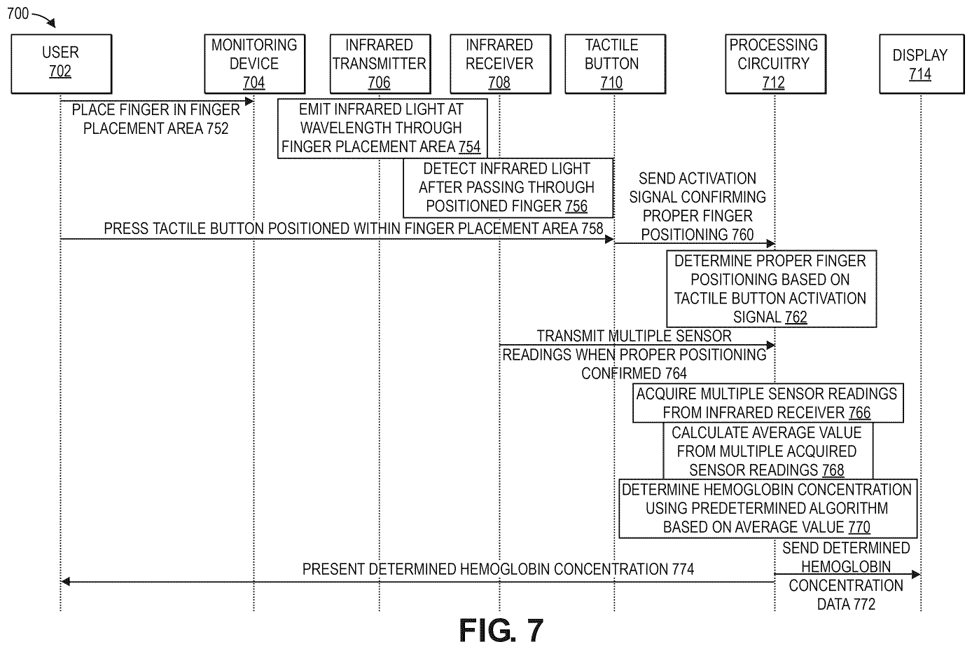

FIG. 7 illustrates a transaction sequence 700 representing a non-invasive hemoglobin monitoring method according to one embodiment. The diagram includes several elements, such as a user 702, a monitoring device 704, an infrared transmitter 706, an infrared receiver 708, a tactile button 710, processing circuitry 712, and a display 714. The process begins when the user 702 places their finger in a finger placement area in step 752. The infrared transmitter 706 emits infrared light a wavelength (such as 880 nm) through the finger placement area in step 754. The infrared receiver 708 detects the infrared light after passing through the positioned finger at 756.

The tactile button 710 registers when pressed within the finger placement area in step 758. The processing circuitry 712 sends an activation signal confirming proper finger positioning at 760. The processing circuitry 712 then determines proper finger positioning based on the tactile button activation signal in step 762. When proper positioning is confirmed, the processing circuitry 712 transmits multiple sensor readings at 764. The processing circuitry 712 acquires multiple sensor readings from the infrared receiver 708 in step 766, calculates an average value from the multiple acquired sensor readings in step 768, and determines hemoglobin concentration using a predetermined algorithm based on the average value in step 770. The processing circuitry 712 sends the determined hemoglobin concentration data in step 772 to the display 714, which presents the determined hemoglobin concentration in step 774.

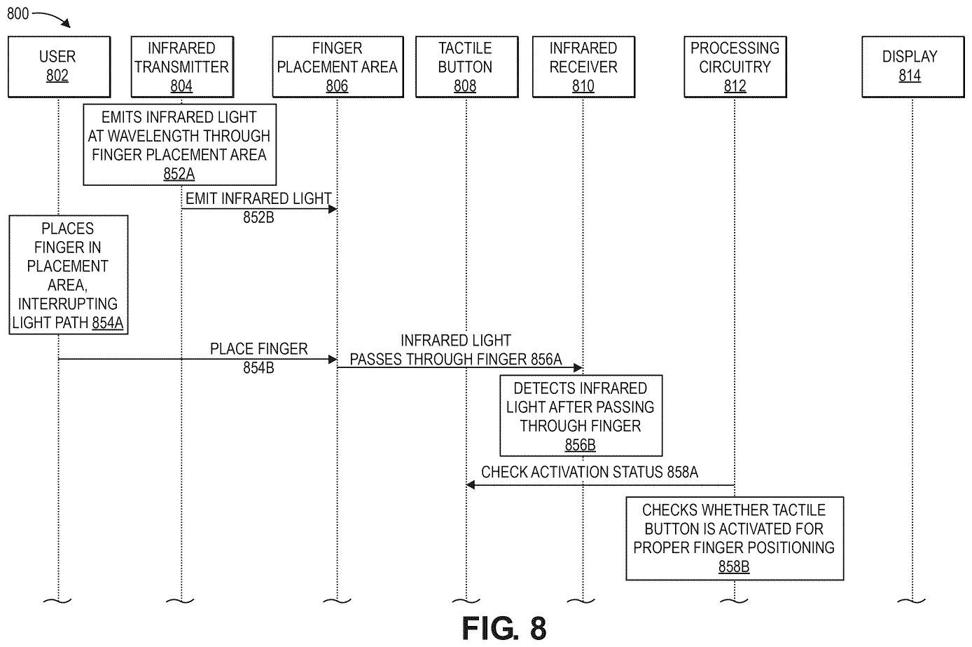

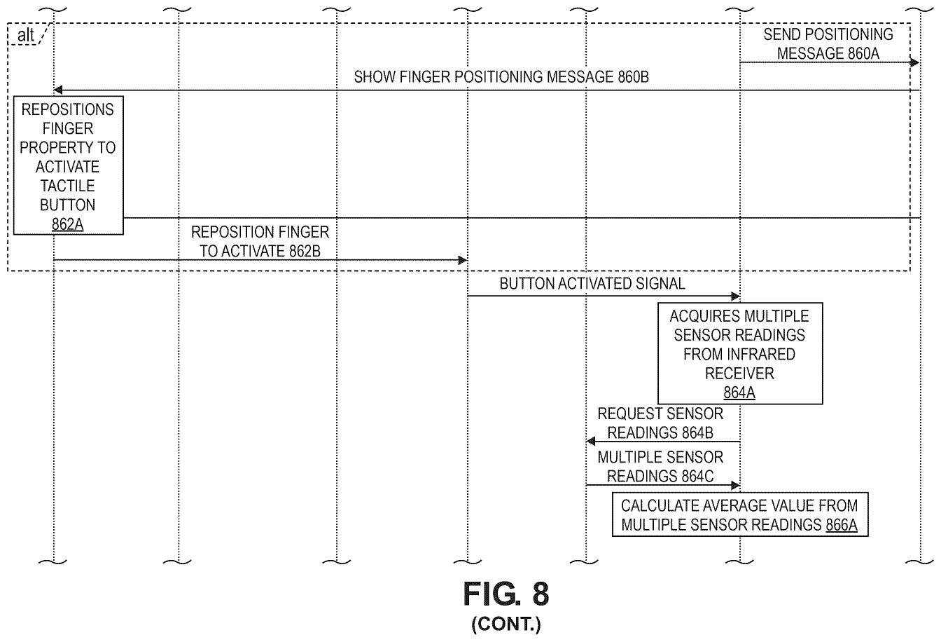

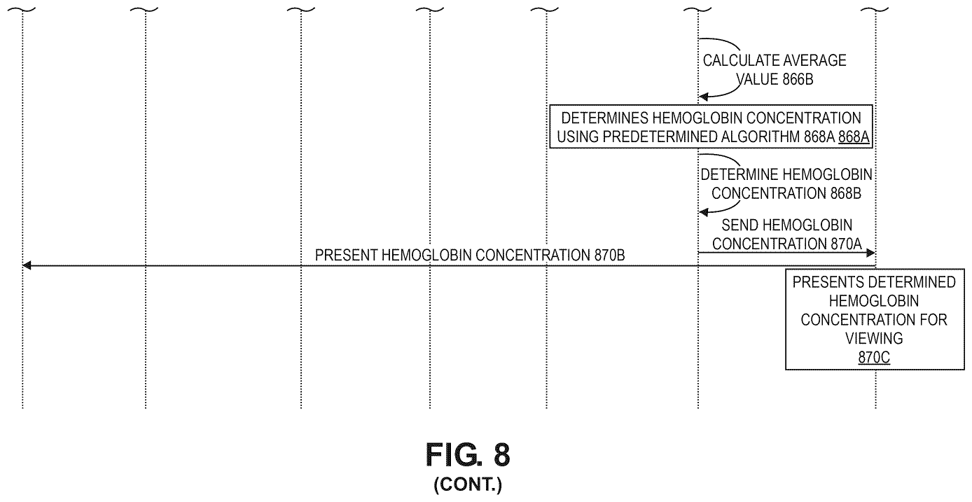

FIG. 8 illustrates a transaction sequence 800 representing a non-invasive hemoglobin monitoring method according to one embodiment. The diagram includes several elements, including a user 802, an infrared transmitter 804, a finger placement area 806, a tactile button 808, an infrared receiver 810, processing circuitry 812, and a display 814. The process begins when the infrared transmitter 804 emits infrared light a wavelength (such as 880 nm) through the finger placement area 806 at 852A and 852B. The user 802 places their finger in the finger placement area 806, interrupting the light path at 854A and 854B. The infrared light passes through the finger at 856A and 856B, reaching the infrared receiver 810. The processing circuitry 812 checks activation status at 858A and 858B, determining whether the tactile button 808 is activated for proper finger positioning. When proper positioning is confirmed, the processing circuitry 812 sends a positioning message at 860A and 860B to the Display 814.

The user 802 repositions their finger properly to activate the tactile button 808 at 862A and 862B. After proper positioning, a button activated signal is sent to the processing circuitry 812. The processing circuitry 812 then acquires multiple sensor readings from the infrared receiver 810 at 864A and 864B. The Processing circuitry 812 calculates an average value from the multiple sensor readings at 866A and 866B, and determines hemoglobin concentration using a predetermined algorithm at 868A and 868B. Finally, the hemoglobin concentration is sent to the display 814 at 870A, 870B and 870C, where it is presented for viewing.

FIG. 9 illustrates a block diagram of a non-invasive hemoglobin monitoring device housing 900 according to one embodiment. The device housing 900 includes a top enclosure containing a main printed circuit board (PCB) 904, which connects to a display PCB 906 and a transmitter PCB 908. The bottom enclosure of the device housing 900 contains a battery 912, a receiver PCB 914, and a button PCB 916. A finger placement area 918 is positioned to enable interaction with the device components. The main PCB 904 provides connections to coordinate operations between the display PCB 906, transmitter PCB 908, receiver PCB 914, and button PCB 916. The battery 912 supplies power to the electronic components within the device housing 900. The arrangement of components within the device housing 900 facilitates the optical measurement path between the transmitter PCB 908 and receiver PCB 914 through the finger placement area 918.

FIG. 10 illustrates a signal processing architecture 1000 of an embodiment of the novel smart non-invasive hemoglobin monitoring device according to one embodiment. The architecture 1000 includes optical sensing components comprising an infrared (IR) transmitter 1004 and IR receiver 1006. The processing unit 1008 contains a microcontroller 1010, Analog-to-Digital Convertor (ADC) circuit 1012, and algorithm engine 1014. The power management section includes a battery system 1018 and voltage regulator 1020. A user interface 1022 and tactile button 1024 are connected to the processing unit 1008. The IR transmitter 1004 and IR receiver 1006 are positioned to create an optical measurement path through a finger placement area. The microcontroller 1010 connects to the ADC circuit 1012 for signal conversion and processing. The algorithm engine 1014 processes the digitized signals to determine hemoglobin concentration. The battery system 1018 provides power through the voltage regulator 1020 to the system components. The user interface 1022 displays measurement results and system status information, while the tactile button 1024 enables user input for measurement control.

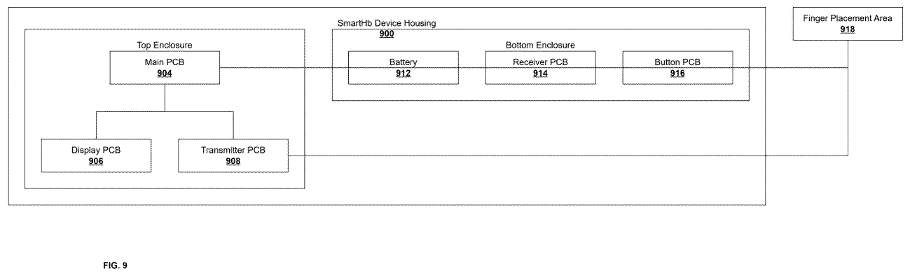

FIG. 11 illustrates an exploded view of a signal processing architecture 1100 of an embodiment of the novel smart non-invasive hemoglobin monitoring device according to one embodiment. The architecture 1100 includes a top portion containing optical sensing components 1102 and an IR transmitter 1104. A central section houses a microcontroller 1110 connected to an ADC circuit 1112. The IR receiver 1148 is positioned to align with the IR transmitter 1104 when the device is assembled. A mounting plate 1150 provides structural support for the components, while a battery compartment 1152 is integrated into the lower portion of the assembly. The components are arranged in a vertical stack configuration that enables proper alignment of the optical sensing elements while maintaining a compact form factor.

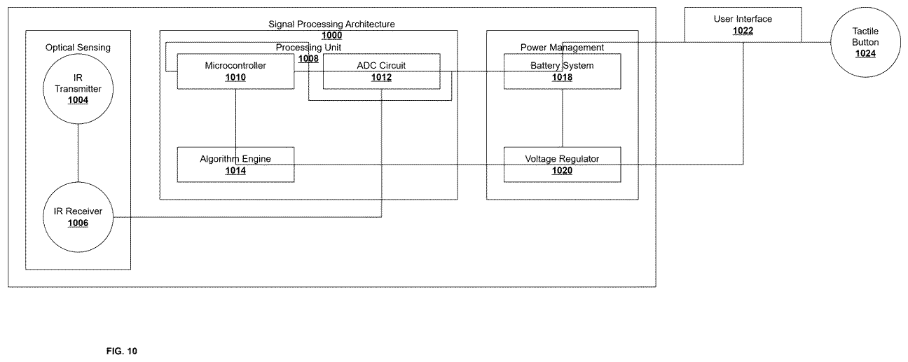

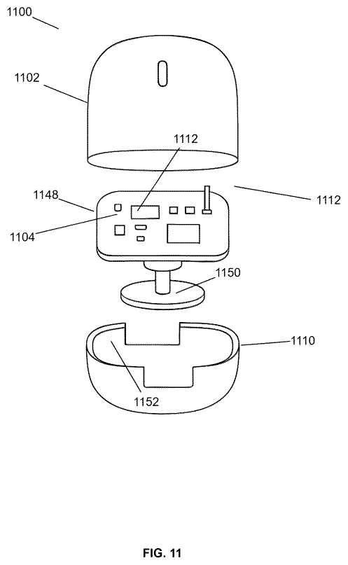

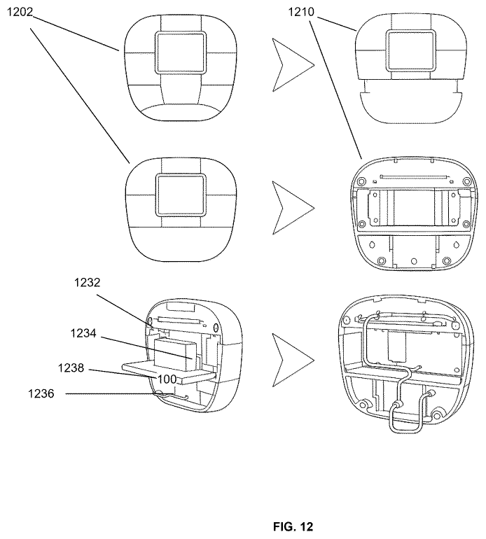

FIG. 12 illustrates various views depicting an assembly sequence of a device housing of an embodiment of the novel smart non-invasive hemoglobin monitoring device according to one embodiment. FIG. 12 depicts a closed view 1202 and an open view 1210 of the device housing and further shows a main PCB 1232 positioned within the housing, followed by a transmitter PCB 1234 and a receiver PCB 1236. The button PCB 1238 is incorporated into the assembly. The bottom enclosure 1210 contains the receiver PCB 1236. The figure illustrates the sequential arrangement of these components through directional arrows indicating the assembly order and spatial relationships between the various PCB components within the housing structure.

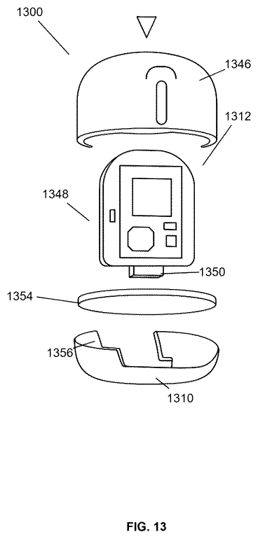

FIG. 13 illustrates an exploded view of a device architecture 1300 of an embodiment of the novel smart non-invasive hemoglobin monitoring device according to one embodiment. The device architecture 1300 includes a display screen 1346 positioned at the top of the assembly. Below the display screen 1346 is a device housing 1348 that contains a microcontroller 1310 and memory storage 1312. The device housing 1348 is supported by a mounting platform 1350. A support plate 1354 is positioned beneath the mounting platform 1350. The base component 1356 forms the bottom portion of the assembly. The components are arranged in a vertical stack configuration that enables proper alignment while maintaining a compact form factor.

FIG. 14 illustrates a diagrammatic representation of a machine 1400 in the exemplary form of a computer system capable of implementing embodiments disclosed in this document. Machine or computing device 1400 employed by smart non-invasive hemoglobin monitoring device may be the same as or similar to or contained within or include smart non-invasive hemoglobin monitoring mechanism to perform or execute one or more methodologies discussed throughout this document. In alternative embodiments, computing device 1400 may be connected (e.g., networked) to other machines either directly, such as via media slot or over a network, such as a cloud-based network, a Local Area Network (LAN), a Wide Area Network (WAN), a Metropolitan Area Network (MAN), a Personal Area Network (PAN), an intranet, an extranet, or the Internet. The machine may operate in the capacity of a server or a client machine in a client-server network environment, or as a peer machine in a peer-to-peer (or distributed) network environment or as a server or series of servers within an on-demand service environment, including an on-demand environment providing multi-tenant database storage services. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

Computing device 1400 includes one or more processors 1402, a main memory 1404 (e.g., read-only memory (ROM), flash memory, dynamic random access memory (DRAM) such as synchronous DRAM (SDRAM) or Rambus DRAM (RDRAM), etc., static memory 1442, such as flash memory, static random access memory (SRAM), volatile but high-data rate RAM, etc.), and a secondary memory 1418 (e.g., a persistent storage device including hard disk drives and persistent multi-tenant data base implementations), which communicate with each other via a bus 1430. Main memory 1404 includes instructions 1424 (such as software 1422 on which is stored one or more sets of instructions 1424 embodying any one or more of the methodologies or functions of smart non-invasive hemoglobin monitoring mechanism at smart non-invasive hemoglobin monitoring device and other figures described herein) which operate in conjunction with processing logic 1426 and processor 1402 to perform the methodologies discussed herein.

Processor 502 represents one or more general-purpose processing devices such as a microprocessor, central processing unit, or the like. More particularly, the processor 1402 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 1402 may also be one or more special-purpose processing devices such as an application specific integrated circuit (ASIC), a field programmable gate array (FPGA), a digital signal processor (DSP), network processor, or the like. Processor 1402 is configured to execute the processing logic 526 for performing the operations and functionality of smart non-invasive hemoglobin monitoring mechanism/device and other figures discussed herein.

Computing device 1400 may further include a network interface device 508, such as a network interface card (NIC). Computing device 500 also may include a user interface 510 (such as a video display unit, a liquid crystal display (LCD), or a cathode ray tube (CRT)), an alphanumeric input device 512 (e.g., a keyboard), a cursor control device 1414 (e.g., a mouse), a signal generation device 1440 (e.g., an integrated speaker), and other devices 1416 like cameras, microphones, integrated speakers, etc. Computing device 1400 may further include peripheral device 1436 (e.g., wireless or wired communication devices, memory devices, storage devices, audio processing devices, video processing devices, display devices, etc.). Computing device 1400 may further include a hardware-based application programming interface logging framework 1434 capable of executing incoming requests for services and emitting execution data responsive to the fulfillment of such incoming requests.

Network interface device 1408 may also include, for example, a wired network interface to communicate with remote devices via network cable 1423, which may be, for example, an Ethernet cable, a coaxial cable, a fiber optic cable, a serial cable, a parallel cable, etc. Network interface device 1408 may provide access to a LAN, for example, by conforming to IEEE 802.11b and/or IEEE 802.11g standards, and/or the wireless network interface may provide access to a personal area network, for example, by conforming to Bluetooth standards. Other wireless network interfaces and/or protocols, including previous and subsequent versions of the standards, may also be supported. In addition to, or instead of, communication via the wireless LAN standards, network interface device 1408 may provide wireless communication using, for example, Time Division, Multiple Access (TDMA) protocols, Global Systems for Mobile Communications (GSM) protocols, Code Division, Multiple Access (CDMA) protocols, and/or any other type of wireless communications protocols.

The secondary memory 1418 may include a machine-readable storage medium (or more specifically a machine-accessible storage medium) 1431 on which is stored one or more sets of instructions (e.g., software 1422) embodying any one or more of the methodologies or functions of smart non-invasive hemoglobin monitoring mechanism/device and other figures described herein. Software 1422 may also reside, completely or at least partially, within the main memory 1404, such as instructions 1424, and/or within the processor 1402 during execution thereof by the computer system 1400, the main memory 1404 and the processor 1402 also constituting machine-readable storage media. Software 1422 may further be transmitted or received over network 1420 via the network interface card 1408. Machine-readable storage medium 1431 may include transitory or non-transitory machine-readable storage media.

Portions of various embodiments may be provided as a computer program product, which may include a computer-readable medium having stored thereon computer program instructions, which may be used to program a computer (or other electronic devices) to perform a process according to the embodiments. The machine-readable medium may include, but is not limited to, floppy diskettes, optical disks, compact disk read-only memory (CD ROM), and magneto optical disks, ROM, RAM, erasable programmable read-only memory (EPROM), electrically EPROM (EEPROM), magnet or optical cards, flash memory, or other type of media/machine readable medium suitable for storing electronic instructions.

Modules 1444 relating to and/or include components and other features described herein (for example in relation to smart non-invasive hemoglobin monitoring mechanism at smart non-invasive hemoglobin monitoring device) can be implemented as discrete hardware components or integrated in the functionality of hardware components such as ASICS, FPGAS, DSPs or similar devices. In addition, modules 1444 can be implemented as firmware or functional circuitry within hardware devices. Further, modules 1444 can be implemented in any combination of hardware devices and software components.

The techniques shown in the figures can be implemented using code and data stored and executed on one or more electronic devices (e.g., an end station, a network element). Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals-such as carrier waves, infrared signals, digital signals). In addition, such electronic devices typically include a set of one or more processors coupled to one or more other components, such as one or more storage devices (non-transitory machine-readable storage media), user input/output devices (e.g., a keyboard, a touchscreen, and/or a display), and network connections. The coupling of the set of processors and other components is typically through one or more buses and bridges (also termed as bus controllers). Thus, the storage device of a given electronic device typically stores code and/or data for execution on the set of one or more processors of that electronic device. Of course, one or more parts of an embodiment may be implemented using different combinations of software, firmware, and/or hardware.

FIG. 15 illustrates a block diagram of a power management system 1500 of an embodiment of the novel smart non-invasive hemoglobin monitoring device according to one embodiment. The power management system 1500 comprises three main subsystems: a battery system, voltage regulation, and power distribution. The battery system includes a lithium battery 1504 that connects to a charging circuit 1506 and a protection circuit 1508. The voltage regulation subsystem receives power from the protection circuit 1508 and includes a boost converter 1512 and a linear regulator 1514. The boost converter 1512 connects to the linear regulator 1514, which provides regulated voltage outputs. The power distribution subsystem receives regulated power from the linear regulator 1514 and distributes it to three separate power rails: main PCB power 1518, display power 1520, and sensor power 1522. The power flow in the system 1500 follows a hierarchical structure, beginning with the lithium battery 1504 and progressing through protection and regulation stages before reaching the individual power distribution points.

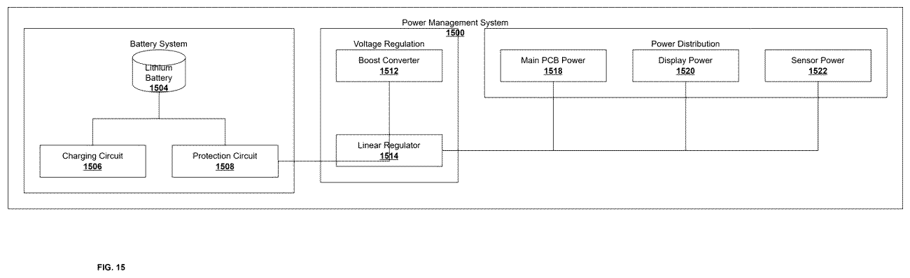

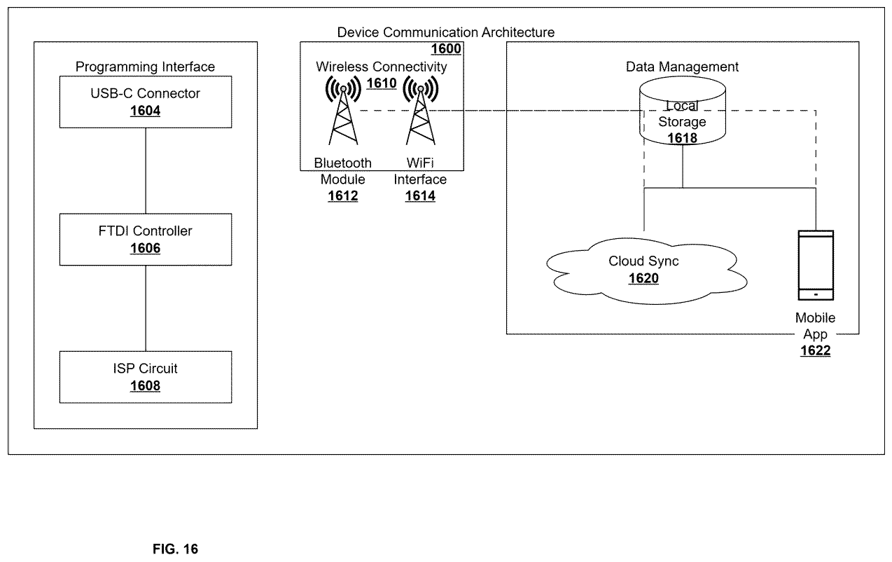

FIG. 16 illustrates a device communication architecture 1600 of an embodiment of the novel smart non-invasive hemoglobin monitoring device according to one embodiment. The architecture 1600 includes a programming interface comprising a Universal Serial Bus-C (USB-C) connector 1604 connected to a Future Technology Devices International (FTDI) controller 1606, which in turn connects to an Internet Service Provider (ISP) circuit 1608. The wireless connectivity 1610 section incorporates a Bluetooth module 1612 and a WiFi interface 1614 for wireless communication capabilities. The data management portion includes local storage 1618 that connects to cloud sync 1620 functionality. A mobile app 1622 interfaces with the data management components. The architecture shows the interconnections between the programming interface, wireless connectivity, and data management components through dashed lines indicating communication pathways between these subsystems.

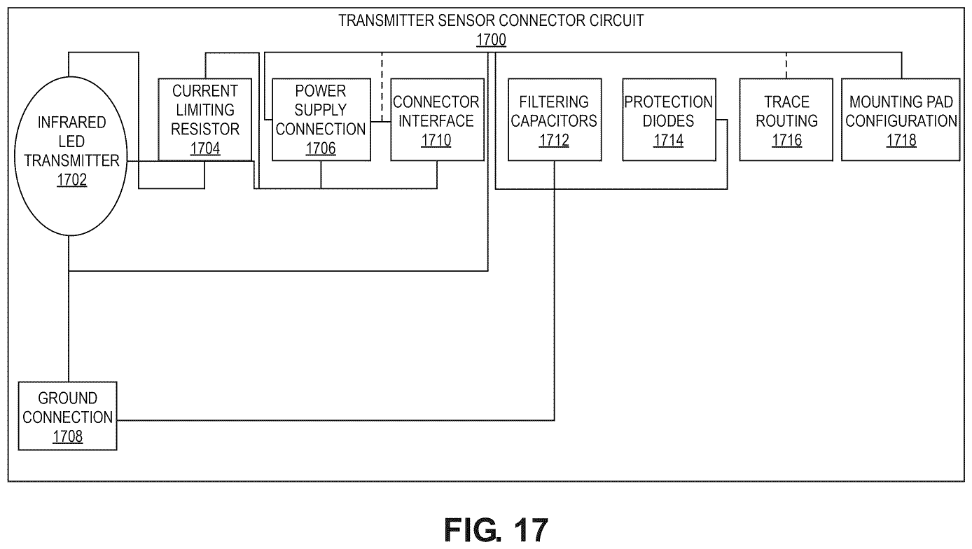

FIG. 17 illustrates a transmitter sensor connector circuit schematic 1700 for the novel smart non-invasive hemoglobin monitoring device according to one embodiment. The circuit schematic 1700 includes an infrared LED transmitter 1702 positioned as the primary light emission component. The infrared LED transmitter 1702 connects to a current limiting resistor 1704 with a value of 220 ohms that regulates the current flow through the LED to prevent damage and ensure stable light output. A power supply connection 1706 provides 5 volts (5V) direct current (DC) power to energize the infrared LED transmitter 1702 through the current limiting resistor 1704. The circuit includes a ground connection 1708 that completes the electrical circuit and provides a reference voltage level for proper operation. A connector interface 1710 enables the transmitter circuit to interface with the main processing circuitry through standardized electrical connections. The transmitter sensor connector circuit schematic 1700 incorporates filtering capacitors 1712 with values of 0.1 microfarads that reduce electrical noise and voltage fluctuations in the power supply lines. Protection diodes 1714 provide reverse polarity protection and prevent damage from voltage spikes or incorrect connections. The circuit layout includes trace routing 1716 that minimizes electromagnetic interference and ensures reliable signal transmission between components. A mounting pad configuration 1718 enables secure physical attachment of the infrared LED transmitter 1702 to the circuit board while maintaining proper thermal management. The transmitter sensor connector circuit schematic 1700 demonstrates the electrical connections and component relationships required for reliable infrared light emission in the hemoglobin monitoring system, with appropriate current regulation, power distribution, and protection mechanisms to ensure stable operation across varying environmental conditions and usage scenarios.

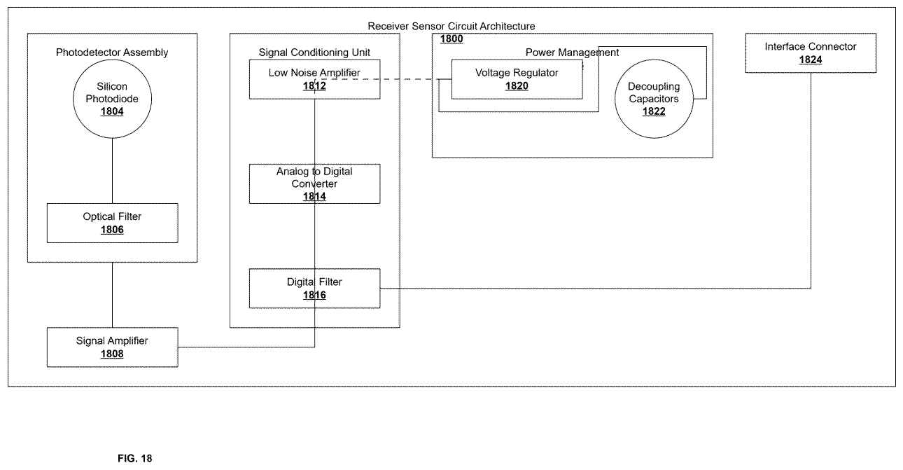

FIG. 18 depicts a receiver sensor connector circuit schematic 1800 for the novel smart non-invasive hemoglobin monitoring device according to one embodiment. The receiver sensor circuit architecture 1800 comprises a photodetector assembly 1802 that serves as the primary optical sensing component for detecting infrared light transmitted through finger tissue during hemoglobin measurement operations. The photodetector assembly 1802 incorporates a silicon photodiode 1804 specifically selected for high sensitivity at the wavelength, such as 880 nm, used by the infrared transmitter. The silicon photodiode 1804 connects to an optical filter 1806 that selectively passes the desired wavelength while blocking ambient light and other optical interference sources that could compromise measurement accuracy. The photodetector assembly 1802 interfaces with a signal amplifier 1808 that conditions the electrical signals generated by the photodiode for subsequent processing. The signal conditioning unit 1810 includes a low noise amplifier 1812 that amplifies the photodetector signals while minimizing noise contributions that could affect measurement precision. The amplified signals are processed through an analog to digital converter 1814 that converts the analog photodetector signals into digital values suitable for computational analysis by the processing circuitry. A digital filter 1816 removes high-frequency noise components and artifacts from the digitized signals before transmission to the main processing unit. The power management 1818 section includes a voltage regulator 1820 that provides stable power supply voltages to the sensitive analog circuits within the receiver assembly. Decoupling capacitors 1822 filter power supply noise and voltage fluctuations that could interfere with the photodetector signals. An interface connector 1824 enables the receiver circuit to communicate with the main processing circuitry through standardized electrical connections that maintain signal integrity and minimize electromagnetic interference during data transmission operations.

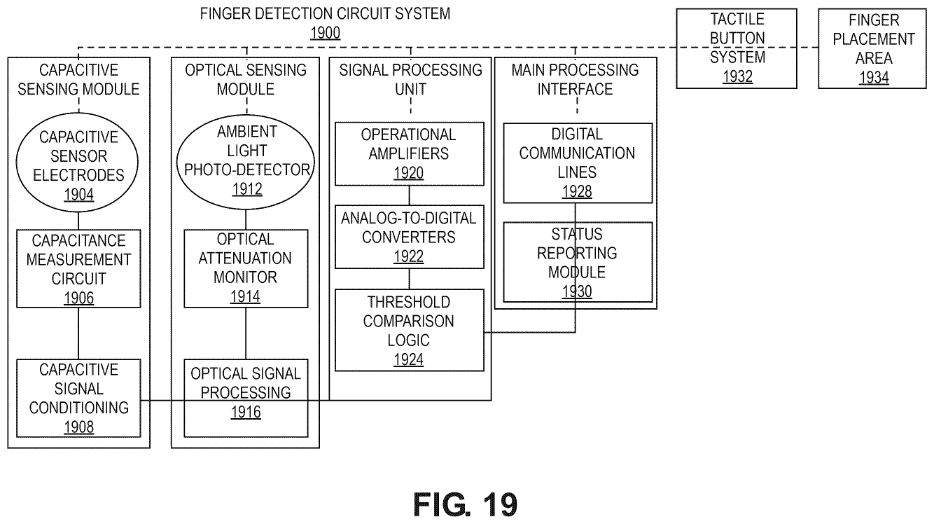

FIG. 19 illustrates a finger detection circuit system 1900 of an embodiment of the novel smart non-invasive hemoglobin monitoring device according to one embodiment. The system 1900 includes a capacitive sensing module comprising capacitive sensor electrodes 1904 connected to a capacitance measurement circuit 1906. The capacitance measurement circuit 1906 connects to capacitive signal conditioning 1908 for processing the sensor signals. An optical sensing module includes an ambient light photodetector 1912 coupled to an optical attenuation monitor 1914, which connects to optical signal processing 1916. A signal processing unit includes operational amplifiers 1920 and analog-to-digital converters 1922 that process signals from both sensing modules. The processed signals feed into threshold comparison logic 1924 that determines proper finger placement. Digital communication lines 1928 connect the processing components to a status reporting module 1930. The system 1900 incorporates a tactile button system 1932 positioned within a finger placement area 1934. The main processing interface includes digital communication lines 1928 that enable data transfer between components. The arrangement of components within the finger detection circuit system 1900 facilitates detection and validation of proper finger positioning through multiple sensing technologies working in coordination.

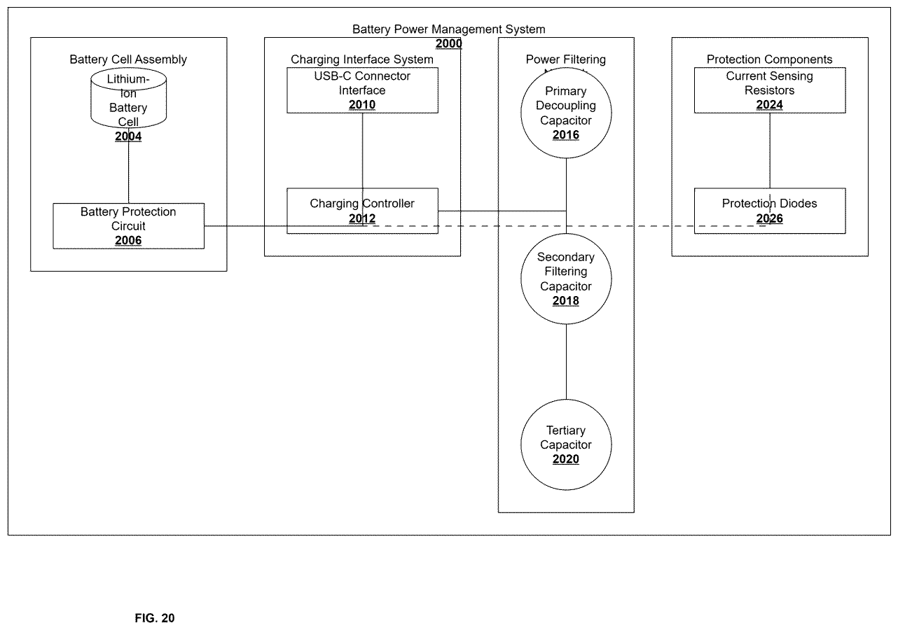

FIG. 20 illustrates a battery power management system 2000 of an embodiment of the novel smart non-invasive hemoglobin monitoring device according to one embodiment. The system 2000 includes a battery cell assembly comprising a lithium-ion battery cell 2004 connected to a battery protection circuit 2006. A charging interface system 2010 includes a USB-C connector interface that connects to a charging controller 2012. The power filtering section contains a primary decoupling capacitor 2016, a secondary filtering capacitor 2018, and a tertiary capacitor 2020. The protection components include current sensing resistors 2024 and protection diodes 2026. The components are arranged in a hierarchical structure, with power flow beginning at the battery cell 2004 and progressing through protection and regulation stages before reaching individual power distribution points.

FIG. 21 illustrates a 5 volts booster circuit system 2100 of an embodiment of the novel smart non-invasive hemoglobin monitoring device according to one embodiment. The system includes an input power section with a battery input 2104 connected to an input decoupling capacitor 2106. A voltage conversion core 2110 contains, for example, an MT3608 Boost Converter that connects to an energy storage inductor 2112 and an SS34 Schottky Diode 2114. The output regulation section includes an output filter capacitor 2118 and a feedback resistor network 2120, which work together to maintain a regulated 5V output 2122. The protection circuitry 2124 incorporates overcurrent protection 2126 and thermal protection 2128 components to safeguard the circuit during operation. The components are arranged in a sequential configuration that enables voltage conversion from the battery input to a regulated 5V output while providing protection against electrical and thermal stress conditions.

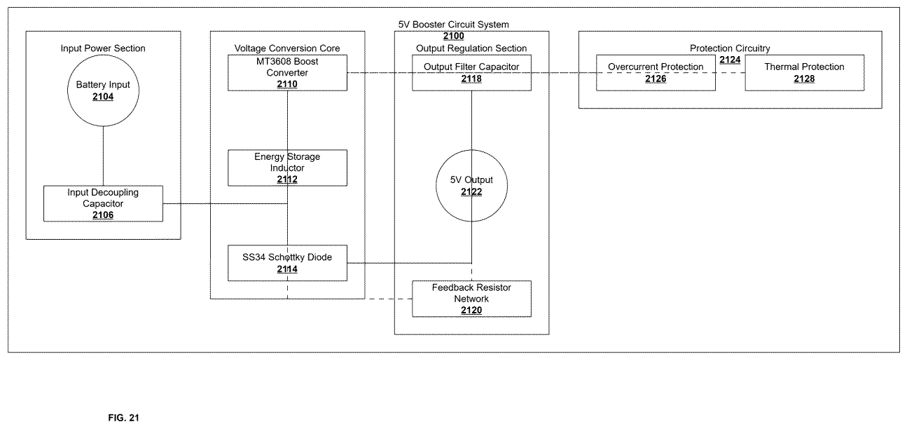

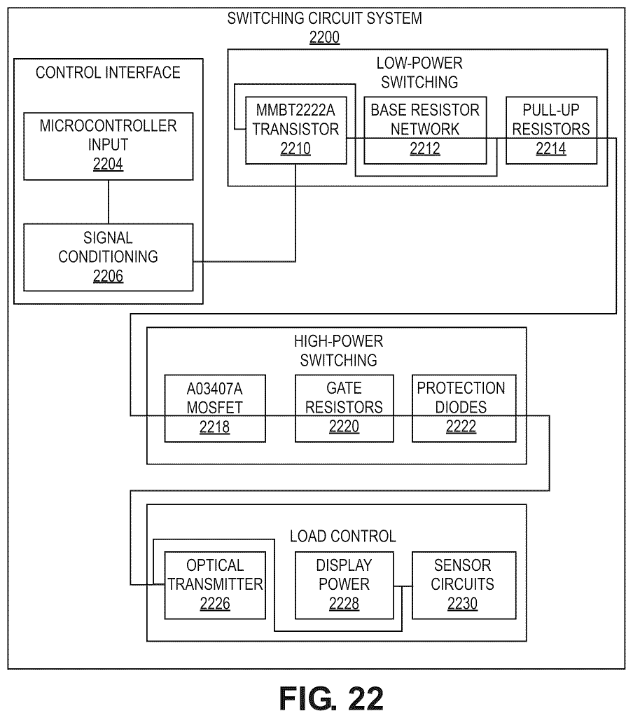

FIG. 22 illustrates a switching circuit system 2200 of an embodiment of the novel smart non-invasive hemoglobin monitoring device according to one embodiment. The system includes a control interface with a microcontroller input 2204 connected to capacitive touch sensors 2206. The capacitive touch sensors 2206 connect to signal fusion processing 2210, which processes input signals from multiple sensing elements. The signal fusion processing 2210 feeds into sensor data aggregation 2212, which combines data from various sensing sources. A quality assessment engine 2214 evaluates the processed sensor data. The feedback control system 2218 manages system responses based on sensor inputs and quality assessments. Gate resistors 2220 provide signal conditioning for the control elements. A tactile feedback generator 2222 produces haptic responses based on system states. The measurement control interface 2226 coordinates with a hemoglobin processing unit 2228, which processes measurement data. Sensor circuits 2230 provide input signals to the system. The components are arranged in a hierarchical structure that enables coordinated operation of low-power switching and high-power switching elements through the control interface.

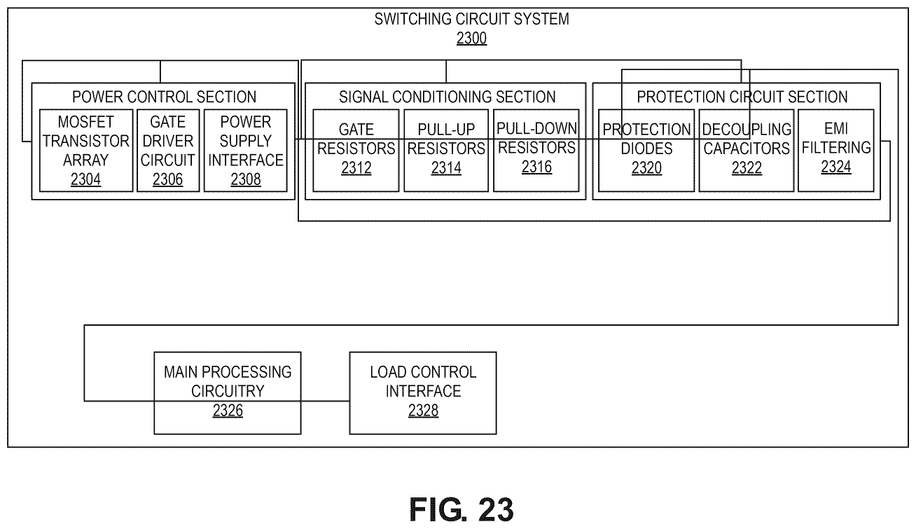

FIG. 23 illustrates a switching circuit component schematic of an embodiment of the novel smart non-invasive hemoglobin monitoring device according to one embodiment. The schematic includes a Metal-Oxide-Semiconductor Field Effect Transistor (MOSFET) Transistor Array 2304 connected to a gate driver circuit 2306. The gate driver circuit 2306 interfaces with a charging interface system 2308, which connects to a charging controller 2312. The power filtering network 2314 contains a primary decoupling capacitor 2316 and a tertiary capacitor 2320 for power supply noise reduction. Protection components 2322 provide circuit protection functionality, working in conjunction with Electromagnetic Interference (EMI) filtering 2324 and protection diodes 2326. The load control interface 2328 manages power distribution to various circuit elements. The components are arranged to provide power control and signal switching capabilities while maintaining protection against electrical transients and electromagnetic interference.

FIG. 24 illustrates an optical sensing system 2400 of an embodiment of the novel smart non-invasive hemoglobin monitoring device according to one embodiment. The optical sensing system 2400 includes an infrared transmitter module 2402 and an infrared receiver module 2410. The infrared transmitter module 2402 contains a light emitting diode (LED) 2404 (e.g., 880 nm LED) connected to a current driver circuit 2406, and an optical lens assembly 2408. The infrared receiver module 2410 comprises a photodetector 2412 coupled to a signal amplifier 2414, and an optical filter 2416. The optical sensing system 2400 includes a tactile button 2420 and positioning guides 2422 for finger placement. The infrared transmitter module 2402 and infrared receiver module 2410 are arranged to create an optical measurement path through which light from the LED 2404 passes through to the photodetector 2412. The current driver circuit 2406 provides regulated power to the LED 2404, while the signal amplifier 2414 processes the signals received by the photo detector 2412. The optical filter 2416 selectively passes desired wavelengths to the photodetector 2412. The positioning guides 2422 work in conjunction with the tactile button 2420 to facilitate proper finger placement during measurement operations.

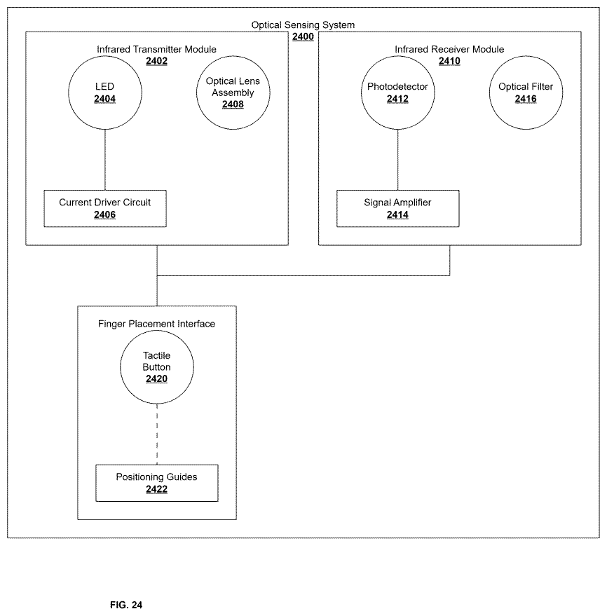

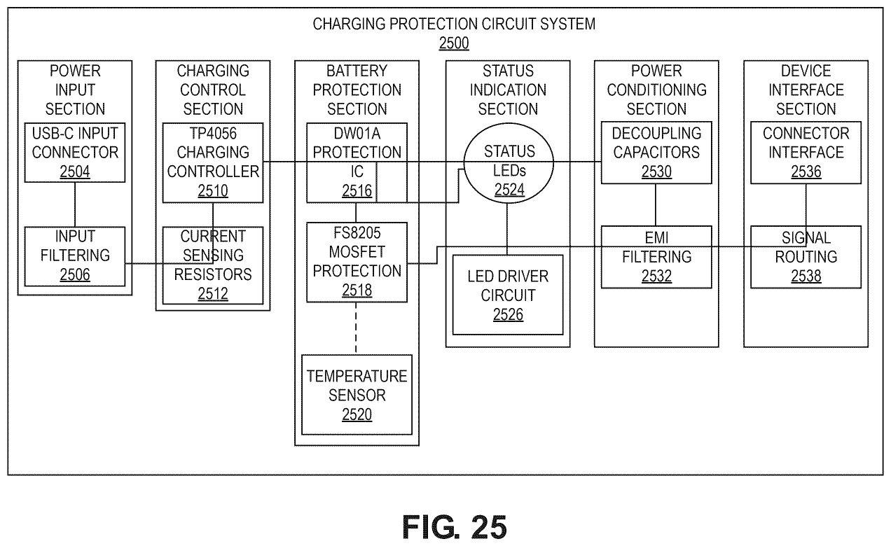

FIG. 25 illustrates a charging protection circuit system 2500 of an embodiment of the novel smart non-invasive hemoglobin monitoring device according to one embodiment. The system includes a power input section with a USB-C Input connector 2504 and input filtering 2506. A charging control section contains a charging controller 2510 connected to current sensing resistors 2512. The battery protection section includes a DW01A Protection integrated circuit (IC) 2516 and MOSFET protection 2518. A temperature sensor 2520 monitors thermal conditions during charging operations. The status indication section comprises status LEDs 2524 connected to an LED driver circuit 2526. The power conditioning section contains decoupling capacitors 2530 and EMI filtering 2532. The device interface section includes a connector interface 2536 and signal routing 2538 that enable communication with other system components. The components are arranged in a hierarchical structure that enables power flow from the USB-C input connector 2504 through protection and regulation stages before reaching the battery charging circuits. The DW01A Protection IC 2516 monitors charging conditions while the MOSFET protection 2518 provides overcurrent protection. The status LEDs 2524 indicate charging states through the LED driver circuit 2526, while the EMI filtering 2532 reduces electrical noise in the system.

FIG. 26 illustrates a programming interface system of an embodiment of the novel smart non-invasive hemoglobin monitoring device according to one embodiment. The system includes a USB-C connector interface 2602 that connects to an FTDI controller chip 2604. The FTDI controller chip 2604 interfaces with a signal conditioning circuit 2606, which incorporates protection resistors 2608 and filtering capacitors 2610. The signal conditioning circuit 2606 connects to an isolation circuit 2612 that provides electrical isolation between the programming interface and internal components. The isolation circuit 2612 connects to programming interface pins including a reset line 2616, clock line 2618, and data lines 2620. These programming interface pins connect to a microcontroller unit 2622, which can be programmed by external programming equipment 2624. The system enables firmware programming and device configuration through standardized electrical connections while providing protection against electrical transients and maintaining signal integrity during programming operations.

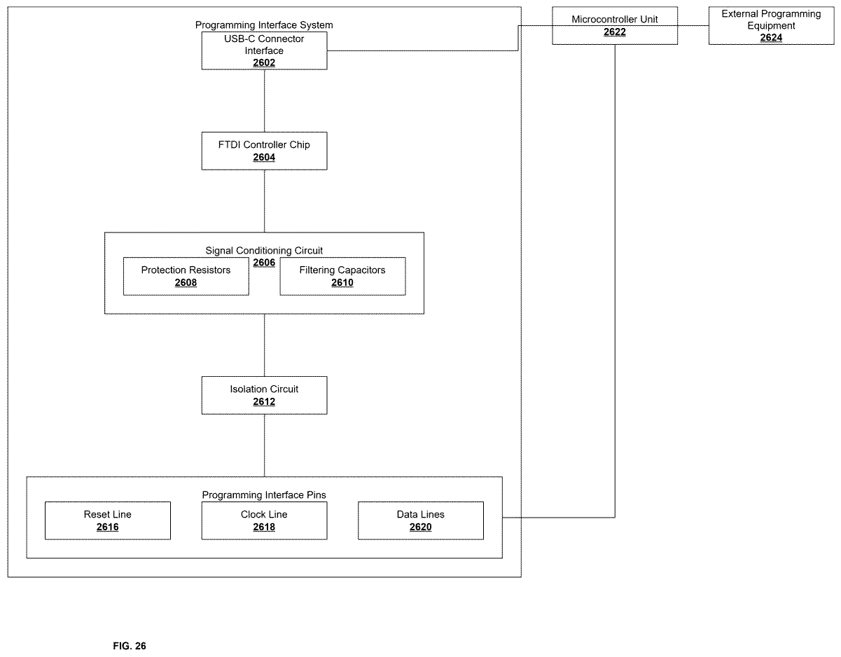

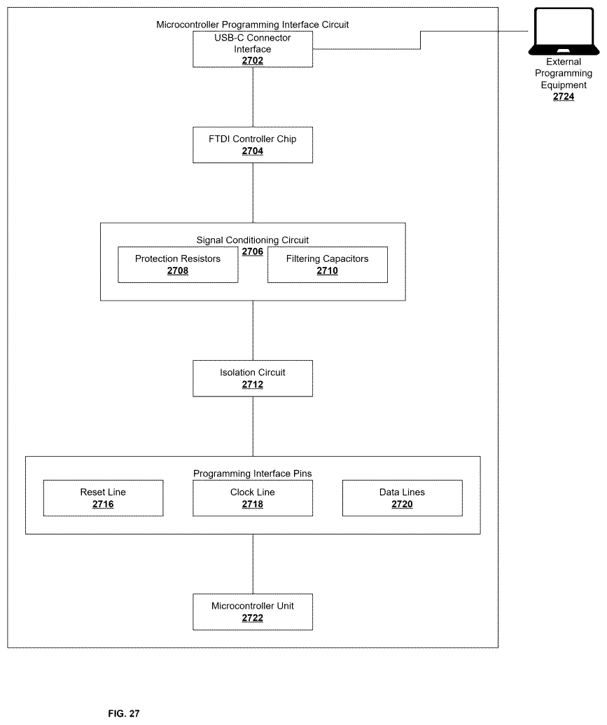

FIG. 27 illustrates a microcontroller programming interface circuit of an embodiment of the novel smart non-invasive hemoglobin monitoring device according to one embodiment. The circuit includes a USB-C Connector Interface 2702 that connects to an FTDI controller chip 2704. The FTDI controller chip 2704 interfaces with a signal conditioning circuit 2706, which incorporates protection resistors 2708 and filtering capacitors 2710. The signal conditioning circuit 2706 connects to an isolation circuit 2712 that provides electrical isolation between the programming interface and internal components. The isolation circuit 2712 connects to programming interface pins including a reset line 2716, clock line 2718, and data lines 2720. These programming interface pins connect to a microcontroller unit 2722, which can be programmed by external programming equipment 2724. The system enables firmware programming and device configuration through standardized electrical connections while providing protection against electrical transients and maintaining signal integrity during programming operations.

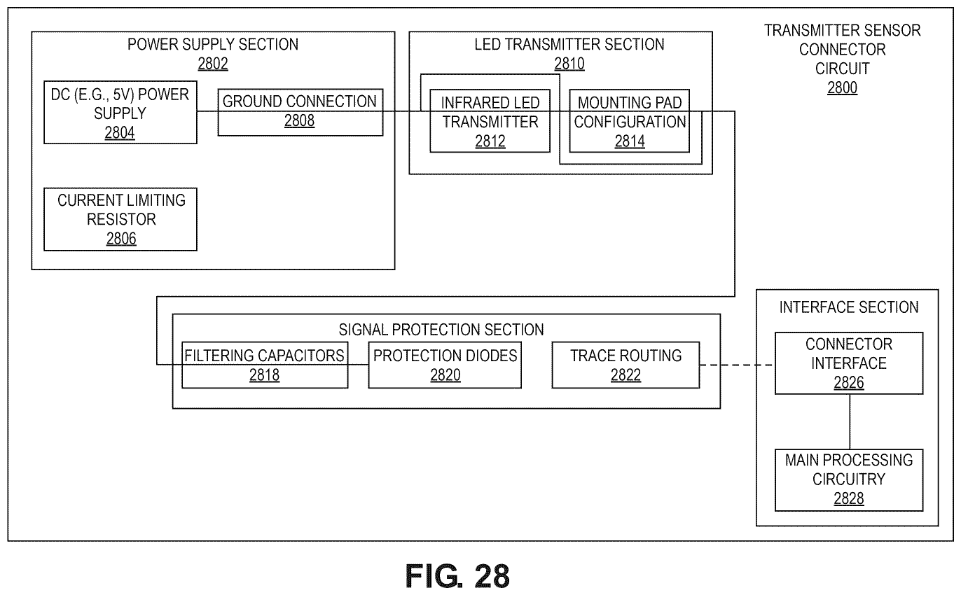

FIG. 28 illustrates a transmitter sensor connector circuit 2800 of an embodiment of the novel smart non-invasive hemoglobin monitoring device according to one embodiment. The circuit 2800 includes a power supply section 2802 containing a 5V Direct Current (DC) power supply 2804 connected to a current limiting resistor 2806 and a ground connection 2808. The LED transmitter section 2810 comprises an infrared LED transmitter 2812 positioned with a mounting pad configuration 2814 that enables secure physical attachment to the circuit board. The circuit 2800 includes filtering capacitors 2818 that reduce electrical noise and voltage fluctuations in the power supply lines. Protection diodes 2820 provide protection against voltage spikes and incorrect connections. The circuit 2800 incorporates trace routing 2822 that facilitates signal transmission between components. A connector interface 2826 enables the transmitter circuit to interface with the main processing circuitry 2828 through standardized electrical connections.

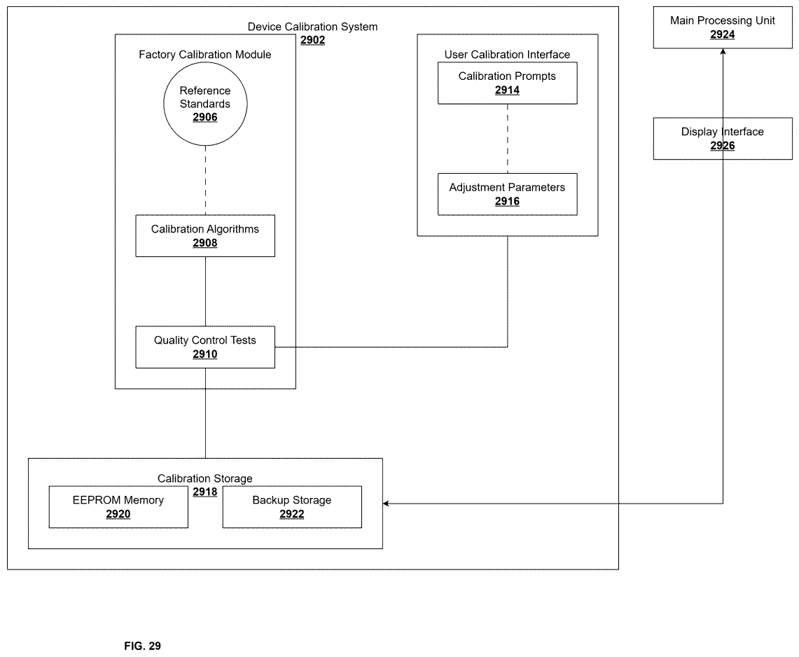

FIG. 29 illustrates a device calibration system 2902 of an embodiment of the novel smart non-invasive hemoglobin monitoring device according to one embodiment. The system 2902 includes a factory calibration module containing reference standards 2906 that connect to calibration algorithms 2908. The calibration algorithms 2908 interface with quality control tests 2910. A user calibration interface incorporates calibration prompts 2914 and adjustment parameters 2916. The system 2902 includes a calibration storage 2918 section that contains Electrical Erasable Programmable Read-Only-Memory (EEPROM) 2920 and backup storage 2922. A main processing unit 2924 connects to the calibration storage 2918 and interfaces with a display interface 2926. The device calibration system 2902 enables data flow between the factory calibration module, user calibration interface, and calibration storage components through interconnected pathways. The main processing unit 2924 coordinates operations between the calibration storage 2918 and display interface 2926 while managing the overall calibration process.

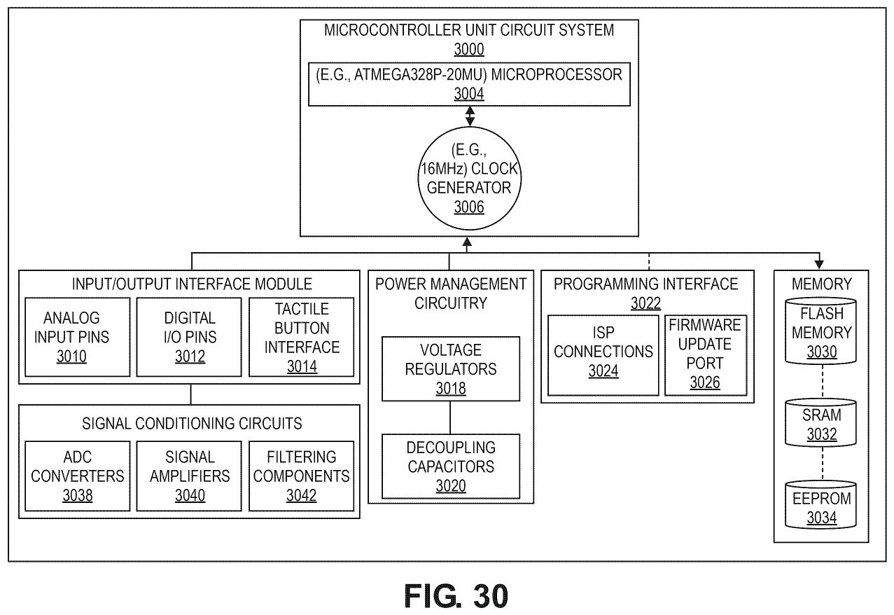

FIG. 30 illustrates a microcontroller unit circuit system 3000 of an embodiment of the novel smart non-invasive hemoglobin monitoring device according to one embodiment. The system 3000 includes a central processing core 3004 connected to a 16 MHz clock generator 3006. The input/output interface module comprises analog input pins 3010, digital input/output (I/O) pins 3012, and a tactile button interface 3014. The power management circuitry includes voltage regulators 3018 and decoupling capacitors 3020. A programming interface 3022 incorporates ISP connections 3024 and a firmware update port 3026. The memory section contains flash memory 3030, Static Random-Access Memory (SRAM) 3032, and EEPROM 3034. Signal conditioning circuits include ADC converters 3038, signal amplifiers 3040, and filtering components 3042. The components are arranged in a hierarchical structure that enables coordinated operation through standardized electrical connections.

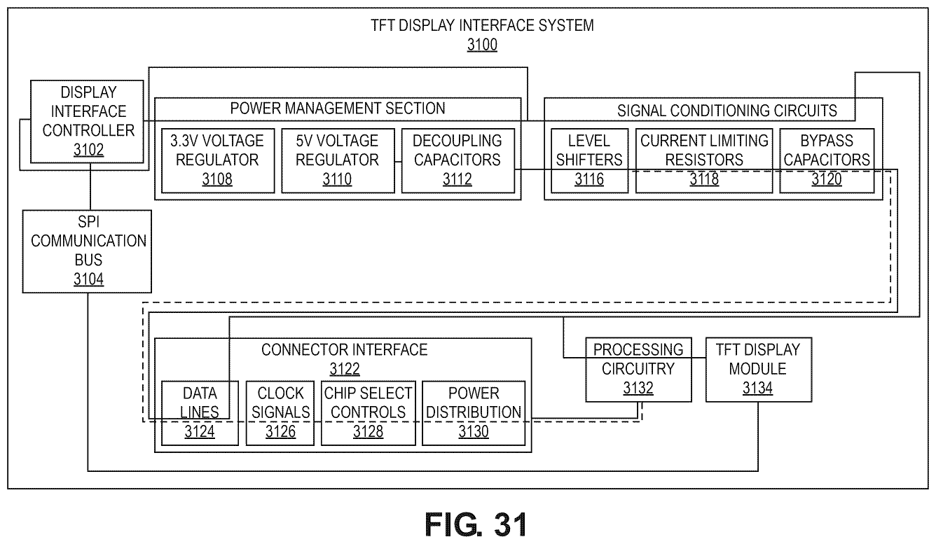

FIG. 31 illustrates a Thin-Film Transistor (TFT) display interface system 3100 of an embodiment of the novel smart non-invasive hemoglobin monitoring device according to one embodiment. The system includes a display interface controller 3102 connected to a Synchronous Serial Peripheral Interface (SPI) communication bus 3104 that enables data transfer between the processing circuitry 3132 and the TFT display module 3134. A power management section 3106 contains a 3.3V voltage regulator 3108 and a 5V voltage regulator 3110, along with decoupling capacitors 3112 for power supply noise reduction. Signal conditioning circuits 3114 incorporate level shifters 3116 that convert signals between different voltage levels. The system includes current limiting resistors 3118 and bypass capacitors 3120 that provide protection and filtering functionality. A connector interface 3122 manages data lines 3124, clock signals 3126, and chip select controls 3128. The power distribution 3130 section delivers regulated power to various components of the system. The processing circuitry 3132 interfaces with the display interface controller 3102 through the SPI communication bus 3104 to control the TFT display module 3134, which presents visual information to users.

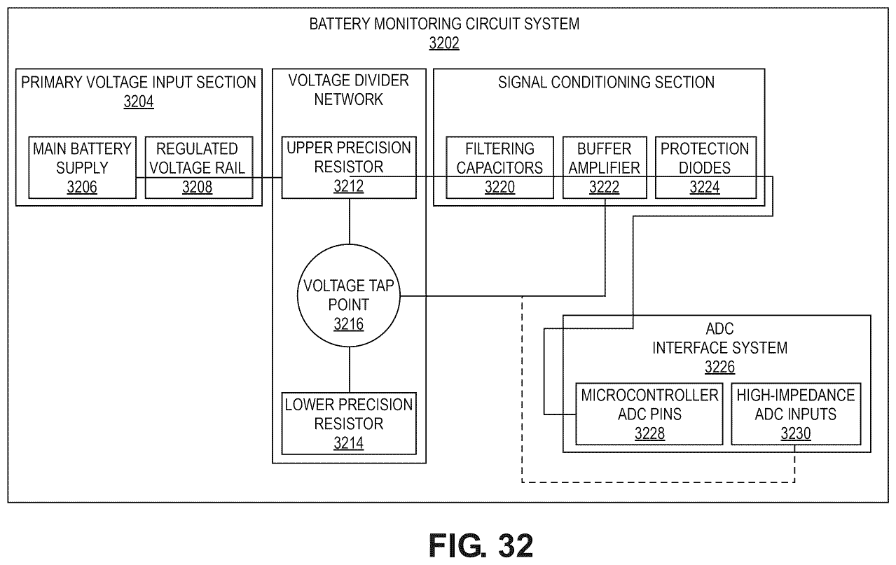

FIG. 32 illustrates a battery monitoring circuit system 3202 of an embodiment of the novel smart non-invasive hemoglobin monitoring device according to one embodiment. The system 3202 includes a primary voltage input section 3204 containing a main battery supply 3206 and a regulated voltage rail 3208. A voltage divider network comprises an upper precision resistor 3212 and a lower precision resistor 3214, with a voltage tap point 3216 positioned between them. The system 3202 incorporates filtering capacitors 3220 connected to a buffer amplifier 3222, followed by protection diodes 3224. An ADC interface section 3226 includes microcontroller ADC pins 3228 and high-impedance ADC inputs 3230. The components are arranged to enable monitoring of battery voltage levels through the voltage divider network, with the filtered and buffered signals being processed through the ADC interface section 3226 for battery level determination.

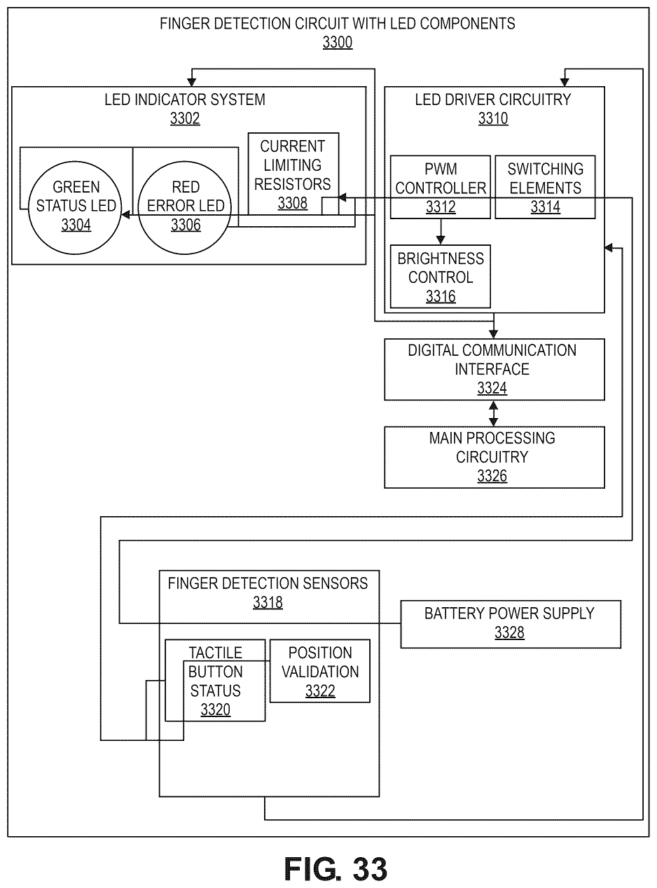

FIG. 33 illustrates a finger detection circuit with LED components of an embodiment of the novel smart non-invasive hemoglobin monitoring device according to one embodiment. The circuit includes an LED indicator system 3302 comprising a green status LED 3304 and a red error LED 3306, connected to current limiting resistors 3308. The LED driver circuitry 3310 contains a pulse-width modulation (PWM) Controller 3312 and switching elements 3314, with a brightness control 3316 module for adjusting LED intensity. The finger detection sensors 3318 include a tactile button status 3320 module and position validation 3322 components. A digital communication interface 3324 connects the sensor components to main processing circuitry 3326. The system is powered by a battery power supply 3328. The LED driver circuitry 3310 controls the operation of the LED indicators through the PWM controller 3312 and switching elements 3314, while the finger detection sensors 3318 monitor finger placement through the tactile button status 3320 and position validation 3322 components. The digital communication interface 3324 facilitates data exchange between the sensing components and main processing circuitry 3326, with power supplied by the battery power supply 3328.

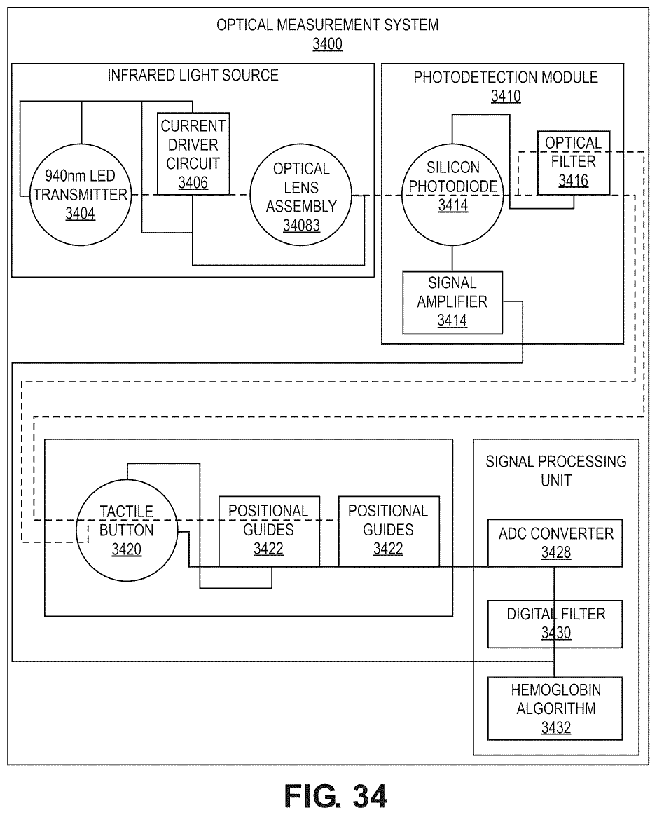

FIG. 34 illustrates a block diagram of an optical measurement system 3400 of an embodiment of the novel smart non-invasive hemoglobin monitoring device according to one embodiment. The optical measurement system 3400 includes, for example, an LED transmitter 3404 (e.g., 880 nm LED transmitter) connected to a current driver circuit 3406 and an optical lens assembly 3408. The photodetection module 3410 contains a silicon photodiode 3412 that connects to a signal amplifier 3414 and an optical filter 3416. A tactile button 3420 and positioning guides 3422 are positioned near an optical sensing window 3424. The system includes signal processing components comprising an ADC converter 3428 connected to a digital filter 3430, which feeds into a hemoglobin algorithm 3432. The LED transmitter 3404 emits light through the optical sensing window 3424, which passes through to the silicon photodiode 3412. The signal amplifier 3414 processes the signals from the silicon photodiode 3412, while the optical filter 3416 selectively passes desired wavelengths. The positioning guides 3422 work in conjunction with the tactile button 3420 to facilitate proper finger placement during measurement operations.

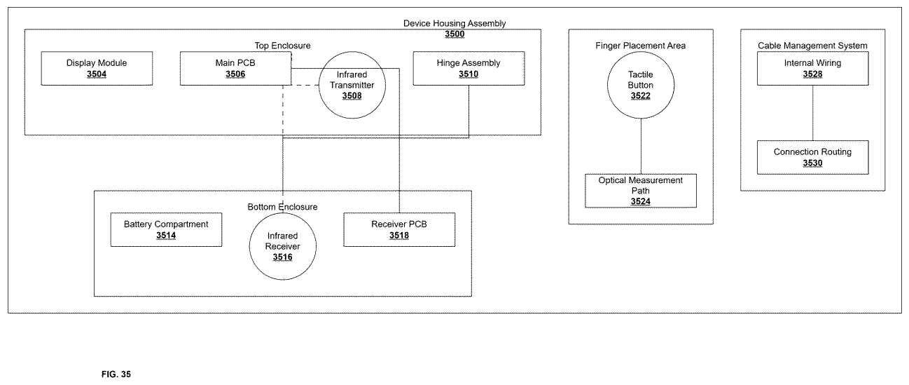

FIG. 35 illustrates a device housing assembly 3500 of an embodiment of the novel smart non-invasive hemoglobin monitoring device according to one embodiment. The device housing assembly 3500 includes a top enclosure containing a display module 3504, a main PCB 3506, and an infrared transmitter 3508. A hinge assembly 3510 connects the top and bottom enclosures. The bottom enclosure contains a battery compartment 3514, an infrared receiver 3516, and a receiver PCB 3518. A tactile button 3522 is positioned near an optical measurement path 3524 that extends between the infrared transmitter 3508 and infrared receiver 3516. Internal wiring 3528 provides electrical connections between components, with connection routing 3530 managing the cable paths within the housing. The arrangement of components enables optical measurement through the optical measurement path 3524 while maintaining a compact form factor.

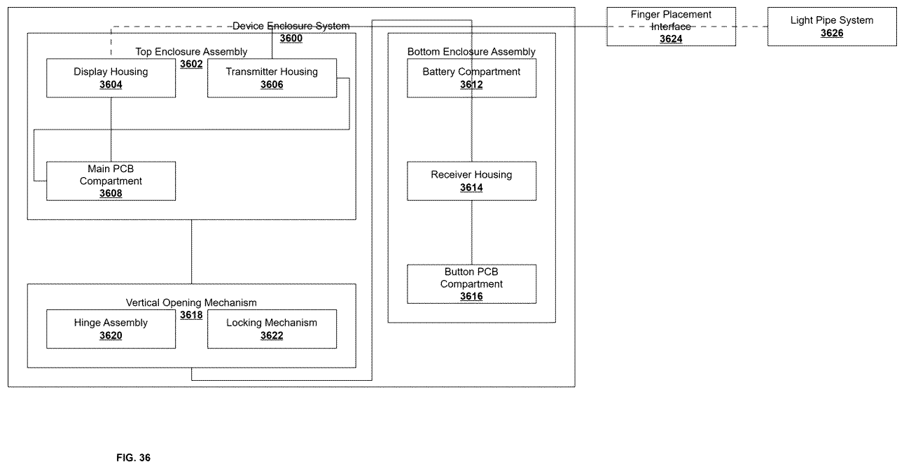

FIG. 36 illustrates a block diagram of a device enclosure system 3600 of an embodiment of the novel smart non-invasive hemoglobin monitoring device according to one embodiment. The device enclosure system 3600 includes a top enclosure assembly 3602 containing a display housing 3604, a transmitter housing 3606, and a main PCB compartment 3608. The display housing 3604 and transmitter housing 3606 are positioned within the top enclosure assembly 3602, with the main PCB compartment 3608 located below these components. The device enclosure system 3600 incorporates a vertical opening mechanism 3618 that includes a hinge assembly 3620 and a locking mechanism 3622. The vertical opening mechanism 3618 connects the top enclosure assembly 3602 to the lower portion of the device. The lower portion contains a battery compartment 3612, a receiver housing 3614, and a button PCB compartment 3616. The battery compartment 3612 connects to the receiver housing 3614, which in turn connects to the button PCB compartment 3616.

A finger placement interface 3624 is positioned between the upper and lower portions of the device. The device enclosure system 3600 also includes a light pipe system 3626 that interfaces with the device components. The arrangement of components within the device enclosure system 3600 creates a structured layout where the display housing 3604 and transmitter housing 3606 are positioned above the main PCB compartment 3608, while the battery compartment 3612, receiver housing 3614, and button PCB compartment 3616 are arranged in the lower portion of the device.

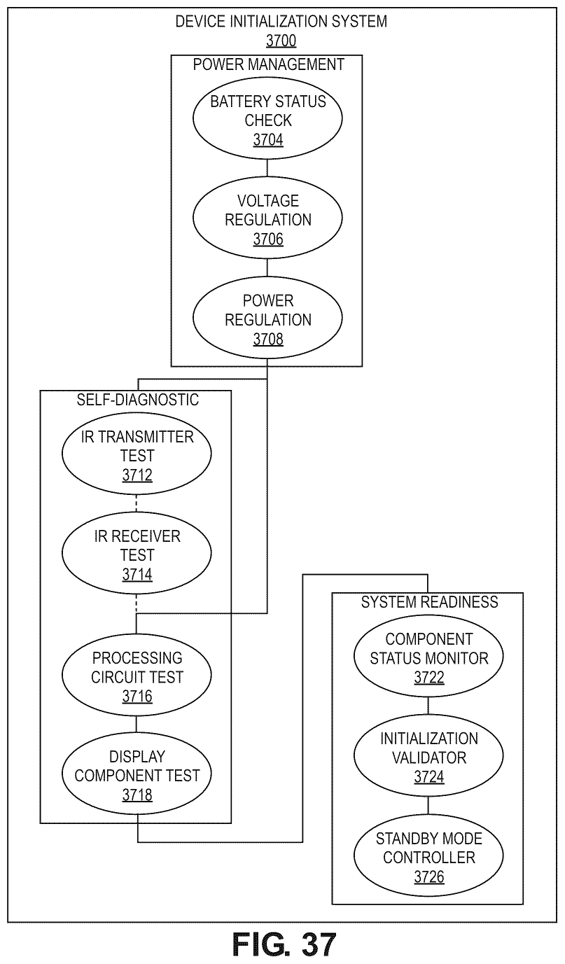

FIG. 37 illustrates a sequence representing a device initialization system 3700 of an embodiment of the novel smart non-invasive hemoglobin monitoring device according to one embodiment. The system 3700 includes a power management section comprising a battery status check 3704, voltage regulation 3706, and power distribution 3708. Following power management, the system 3700 performs a self-diagnostic sequence that includes an IR transmitter test 3712, IR receiver test 3714, processing circuit test 3716, and display component test 3718. After completing the diagnostic tests, the system 3700 proceeds to a system readiness phase where a component status monitor 3722 assesses the operational status of device components. The initialization validator 3724 then verifies that all initialization steps have been completed successfully. Finally, a standby mode controller 3726 manages the device's standby state when initialization is complete. The sequence demonstrates the sequential flow of operations from power management through self-diagnostics to system readiness, incorporating validation steps that ensure proper device functionality before entering standby mode.

### Optical Sensing Components

The optical sensing components of the non-invasive hemoglobin monitoring device comprise an infrared transmitter and an infrared receiver positioned within the housing to create an optical measurement pathway through the finger placement area. The infrared transmitter may be configured to emit light at a specific wavelength (such as 880 nm), which corresponds to an optimal absorption wavelength for hemoglobin detection in biological tissue. This wavelength selection may provide enhanced measurement sensitivity while minimizing interference from other tissue components and ambient light sources.

The infrared transmitter may comprise a light-emitting diode (LED) or laser diode configured to produce stable, consistent light output at the designated wavelength. The transmitter may be positioned within the top enclosure of the housing and aligned with the finger placement area to ensure proper light transmission through the user's finger. The transmitter may include optical focusing elements such as lenses or collimators that concentrate the emitted light into a defined beam pattern, improving measurement accuracy and reducing power consumption.

The infrared receiver may comprise a photodetector or photodiode positioned within the bottom enclosure of the housing and aligned with the infrared transmitter to detect light that has passed through the user's finger. The receiver may be configured to convert received optical signals into electrical signals proportional to the light intensity, enabling subsequent signal processing and hemoglobin concentration calculation. The receiver may include optical filtering elements that selectively pass the wavelength (e.g., 880 nm) while blocking ambient light and other optical interference sources.

The optical path between the transmitter and receiver may be configured to pass through the finger placement area, creating a transmission measurement configuration where hemoglobin concentration is determined based on light absorption according to Beer-Lambert law principles. The optical components may be positioned to maintain a consistent measurement distance and beam alignment, ensuring reproducible measurement conditions across multiple uses and different finger sizes.

### Finger Positioning and Detection Mechanism

The finger positioning and detection mechanism incorporates a tactile button positioned within the finger placement area to provide confirmation of proper finger positioning before measurement initiation. The tactile button may be configured as a pressure-sensitive switch that activates when sufficient downward force is applied by the user's finger. This tactile feedback mechanism may ensure that the finger is properly positioned within the optical measurement path and maintains adequate contact pressure throughout the measurement process.

The tactile button may comprise a mechanical switch element such as a dome switch or membrane switch that provides tactile feedback when activated. The button may be integrated into the finger placement area surface and positioned to align with the optical measurement path between the transmitter and receiver components. The button activation may require a predetermined force threshold that indicates proper finger placement while remaining comfortable for users across different age groups and physical capabilities.

The finger detection system may also incorporate additional sensing mechanisms to verify finger presence and positioning quality. These mechanisms may include capacitive sensors that detect the presence of biological tissue or optical sensors that monitor light transmission levels to confirm adequate finger contact. The combination of tactile button activation and supplementary sensing methods may provide comprehensive finger positioning validation, reducing measurement errors associated with improper finger placement.

The finger positioning mechanism may include visual or audible feedback indicators that guide users through the proper finger placement process. These indicators may activate when the tactile button is pressed and proper finger positioning is achieved, providing confirmation that measurement can proceed. The feedback system may also indicate when finger repositioning is required due to inadequate contact or improper alignment with the optical sensing components.

### Processing Circuitry and Signal Processing

The processing circuitry comprises a microcontroller unit coupled to the infrared receiver and tactile button to coordinate measurement operations and perform hemoglobin concentration calculations. The microcontroller may be configured to monitor the tactile button status and initiate measurement sequences only when proper finger positioning is confirmed through button activation. This control mechanism may prevent measurement attempts when finger positioning is inadequate, improving measurement reliability and reducing false readings.

The processing circuitry may be configured to acquire multiple sensor readings from the infrared receiver when proper finger positioning is confirmed. In some implementations, the system may acquire 20 sensor readings with a 0.5-second delay between each reading to ensure measurement stability and reduce the influence of physiological variations such as pulse or breathing artifacts. The multiple reading approach may provide improved measurement accuracy through statistical averaging and noise reduction.

The signal processing algorithms may include analog-to-digital conversion circuitry that converts the electrical signals from the infrared receiver into digital values suitable for computational processing. The analog-to-digital converter may provide sufficient resolution and sampling rate to capture the subtle variations in light transmission that correspond to hemoglobin concentration differences. The digital signal processing may include filtering algorithms that remove noise and artifacts while preserving the hemoglobin-related signal components.

The processing circuitry may implement a predetermined algorithm that calculates hemoglobin concentration based on the average value of the acquired sensor readings. The algorithm may comprise a compartmental model that selects between different calculation formulas based on the measured signal characteristics. In some cases, the algorithm may select between a first calculation formula and a second calculation formula based on whether the average sensor value is less than 1017 or greater than or equal to 1017, providing optimized calculation methods for different signal ranges.

### Display and User Interface

The display component may comprise a digital display module such as a thin-film transistor (TFT) liquid crystal display (LCD) or organic light-emitting diode (OLED) display configured to present measurement results and system status information to the user. The display may be positioned within the top enclosure of the housing and oriented to provide clear visibility during device operation. The display size and resolution may be selected to accommodate the presentation of numerical hemoglobin values, measurement units, and status messages in a clear, readable format.

The display may be configured to present hemoglobin concentration values in standard medical units such as grams per deciliter (g/dL) or millimoles per liter (mmol/L). The numerical display may include appropriate decimal precision to provide clinically relevant measurement resolution while maintaining display clarity. The display may also include graphical elements such as progress indicators during measurement acquisition or battery status indicators to inform users of device operational status.

The user interface may incorporate additional visual indicators such as light-emitting diodes (LEDs) that provide status information during device operation. These indicators may include power status lights, measurement progress indicators, or error condition warnings. The LED indicators may be positioned on the exterior housing surface and configured to provide clear visibility under various ambient lighting conditions.

The display and user interface components may be controlled by the processing circuitry to provide coordinated feedback during measurement operations. The interface may guide users through the measurement process with sequential prompts and confirmations, ensuring proper device operation and measurement quality. The interface may also provide troubleshooting guidance when measurement errors or positioning problems are detected.

### Validation and Error Handling

The processing circuitry may be configured to validate calculated hemoglobin concentration values by checking whether the results fall within predetermined physiological ranges. The validation process may compare calculated values against a range of 1 to 17.5 grams per deciliter, which encompasses the typical range of hemoglobin concentrations found in healthy and pathological conditions. Values falling outside this range may indicate measurement errors, improper finger positioning, or device malfunction.

When calculated hemoglobin concentrations fall outside the predetermined range, the processing circuitry may be configured to display error messages on the display to inform users of the measurement problem. These error messages may include specific guidance such as “Finger Placement Error” or “Repositioning Required” to help users understand the corrective actions needed. The error handling system may prevent the display of invalid measurement results while providing clear instructions for obtaining accurate measurements.

The validation system may incorporate multiple levels of error checking to ensure measurement quality. Primary validation may check for proper finger positioning through tactile button activation and sensor signal levels. Secondary validation may assess the quality of acquired sensor readings by analyzing signal stability, noise levels, and consistency across multiple measurements. Tertiary validation may evaluate the calculated hemoglobin concentration against physiological limits and measurement repeatability criteria.

The error handling mechanisms may include automatic retry capabilities that prompt users to reposition their finger and attempt measurement again when errors are detected. The system may track the number of measurement attempts and provide escalating guidance or recommendations for device maintenance when repeated measurement failures occur. This comprehensive error handling approach may improve measurement success rates while maintaining measurement accuracy standards.

### Power Management System

The power management system comprises a rechargeable battery power supply and associated charging and regulation circuitry to provide stable electrical power for device operation. The rechargeable battery may comprise lithium-ion or lithium-polymer cells selected for their energy density, cycle life, and safety characteristics. The battery capacity may be sized to support extended operational periods, enabling multiple measurement sessions between charging cycles.

The charging system may incorporate a USB-C connector or wireless charging interface to provide convenient battery recharging capabilities. The charging circuitry may include protection mechanisms such as overcharge protection, overcurrent protection, and thermal monitoring to ensure safe charging operations. The charging system may also include status indicators that inform users of charging progress and battery condition.

The voltage regulation circuitry may provide stable power supply voltages for the various electronic components within the device. The regulation system may include multiple voltage rails to accommodate different component requirements, such as 3.3V for digital circuitry and 5V for optical components. The voltage regulators may be configured to maintain stable output voltages across varying battery charge levels and load conditions.

The power management system may include power-saving features such as automatic shutdown or sleep modes that reduce power consumption during periods of inactivity. These features may extend battery life while ensuring that the device is ready for immediate use when needed. The power management may also include low-battery warnings and automatic shutdown mechanisms to prevent damage from deep discharge conditions.

### Programming and Connectivity Features

The device may include programming interfaces that enable firmware updates and configuration modifications through external connections. The programming interface may comprise a standard connector such as a USB interface or dedicated programming pins that provide access to the microcontroller programming functions. This capability may enable manufacturers to update device firmware, calibration parameters, or operational algorithms after manufacturing.

The programming interface may support in-system programming (ISP) or in-circuit programming (ICP) methods that allow firmware updates without disassembling the device. The programming connections may be protected by covers or seals to prevent contamination while maintaining accessibility for authorized service personnel. The programming interface may also include security features that prevent unauthorized firmware modifications or device tampering.

In some implementations, the device may include wireless connectivity features such as Bluetooth or Wi-Fi capabilities that enable data transfer to external devices or cloud-based systems. These connectivity features may support remote monitoring applications, data logging, or integration with electronic health record systems. The wireless capabilities may be implemented through separate communication modules or integrated into the main processing circuitry.

The connectivity features may include data encryption and authentication mechanisms to protect patient information and ensure data integrity during transmission. The communication protocols may comply with healthcare data security standards and regulations to maintain patient privacy and confidentiality. The connectivity system may also include user consent mechanisms and data management controls that allow users to control data sharing and storage preferences.

### Alternative Embodiments and Configurations

Alternative embodiments of the non-invasive hemoglobin monitoring device may incorporate different optical sensing configurations, such as reflectance-based measurement systems that detect light reflected from tissue surfaces rather than transmitted light. Reflectance-based systems may enable measurements from alternative body locations such as the forehead, earlobe, or wrist, providing flexibility in measurement site selection and accommodating users with finger accessibility limitations.

The device may be configured with multiple wavelength optical sources to provide enhanced measurement accuracy and compensation for tissue variations. Multi-wavelength systems may utilize additional infrared or near-infrared wavelengths that provide complementary information about tissue composition and hemoglobin distribution. The multi-wavelength approach may improve measurement accuracy across diverse patient populations and reduce the influence of confounding factors such as skin pigmentation or tissue thickness.

Alternative housing configurations may incorporate different form factors such as wrist-worn devices, clip-on sensors, or handheld units with probe attachments. These alternative configurations may provide improved portability, hands-free operation, or specialized measurement capabilities for specific clinical applications. The housing design may be adapted to accommodate different measurement sites while maintaining the optical alignment and user interface requirements.

The device may include additional sensing capabilities such as pulse oximetry, heart rate monitoring, or temperature measurement to provide comprehensive physiological assessment in a single device. These multi-parameter monitoring capabilities may enhance the clinical utility of the device while maintaining the compact form factor and ease of use characteristics. The additional sensors may share common optical components or processing circuitry to minimize device complexity and cost.

### Method of Operation

The method of operation for the non-invasive hemoglobin monitoring device begins with device activation and initialization procedures that verify system functionality and prepare the optical sensing components for measurement. The initialization process may include self-diagnostic checks of the optical transmitter, receiver, processing circuitry, and display components to ensure proper operation before measurement attempts.

The measurement process may commence with user finger placement in the designated finger placement area of the device housing. The user may be guided through proper finger positioning through visual or audible prompts displayed on the device interface. The finger positioning process may require activation of the tactile button to confirm adequate finger contact and proper alignment with the optical sensing components.

Upon confirmation of proper finger positioning through tactile button activation, the processing circuitry may initiate the optical measurement sequence by activating the infrared transmitter and beginning data acquisition from the infrared receiver. The measurement process may involve acquiring multiple sensor readings over a predetermined time period to ensure measurement stability and accuracy. The acquired data may be processed through filtering and averaging algorithms to reduce noise and improve measurement precision.

The hemoglobin concentration calculation may be performed using predetermined algorithms that convert the optical measurement data into clinically relevant hemoglobin concentration values. The calculation process may include calibration corrections, temperature compensation, and validation checks to ensure measurement accuracy and reliability. The final hemoglobin concentration result may be displayed on the device screen along with measurement timestamp and quality indicators.

### System Integration and Functionality

The system integration of the non-invasive hemoglobin monitoring device encompasses the coordination of optical sensing, signal processing, user interface, and power management subsystems to provide seamless measurement functionality. The integration architecture may utilize a centralized control approach where the main processing circuitry coordinates the operation of all subsystems through standardized communication interfaces and control protocols.

The optical sensing subsystem may interface with the processing circuitry through analog signal conditioning circuits that amplify, filter, and convert the optical signals into digital format suitable for computational processing. The signal conditioning may include gain control mechanisms that automatically adjust signal levels to optimize measurement dynamic range and accuracy across different finger sizes and tissue characteristics.

The user interface subsystem may provide bidirectional communication between the user and the device through display output and tactile input mechanisms. The interface may implement state-based operation modes that guide users through measurement procedures while providing feedback on measurement progress and results. The interface design may accommodate users with varying technical expertise levels through intuitive operation sequences and clear status indicators.

The power management subsystem may monitor and control electrical power distribution throughout the device while optimizing battery life and ensuring stable operation. The power management may implement dynamic power scaling that adjusts component power consumption based on operational requirements and battery status. The system may also include power-on self-test procedures that verify component functionality before enabling measurement operations.

The optical sensing components of the non-invasive hemoglobin monitoring device comprise an infrared transmitter and an infrared receiver that work in conjunction to create a transmission-based measurement system for determining hemoglobin concentration in biological tissue. The infrared transmitter may be configured to emit light at a wavelength, such as 880 nanometers, which provides optimal absorption characteristics for hemoglobin detection while minimizing interference from other tissue components. The wavelength selection of the given wavelength, such as 880 nanometers, falls within the near-infrared spectrum where hemoglobin exhibits distinct absorption properties that enable accurate concentration measurements through optical transmission analysis.

The infrared transmitter may comprise a light-emitting diode (LED) or laser diode specifically designed to produce stable, monochromatic light output at the designated wavelength. The transmitter component may be positioned within the housing structure to align with the finger placement area, ensuring that emitted light passes through the user's finger tissue during measurement operations. The positioning of the infrared transmitter may be optimized to create a uniform light distribution across the measurement area while maintaining consistent beam characteristics throughout the operational lifetime of the device. The transmitter may include current regulation circuitry that maintains stable light output intensity regardless of temperature variations or battery voltage fluctuations.

The infrared receiver may comprise a photodetector or photodiode element configured to detect and convert optical signals into electrical signals proportional to the received light intensity. The photodetector may be positioned within the housing structure in alignment with the infrared transmitter to create a direct optical path through the finger placement area. The receiver component may incorporate spectral filtering elements that selectively respond to the wavelength (such as 880 nm) while rejecting ambient light and other optical interference sources that could affect measurement accuracy. The photodetector may utilize silicon-based semiconductor technology that provides high sensitivity and low noise characteristics suitable for detecting the subtle variations in light transmission that correspond to hemoglobin concentration differences.

The optical sensing technology employed in the device may operate according to Beer-Lambert law principles, where the absorption of light passing through tissue is proportional to the concentration of absorbing substances, including hemoglobin. The measurement process may involve analyzing the attenuation of the light (e.g., 880 nm light) as the light passes through finger tissue, with greater hemoglobin concentrations resulting in increased light absorption and correspondingly reduced signal levels at the photodetector. The optical sensing system may be calibrated to correlate specific light transmission levels with known hemoglobin concentration values, enabling accurate quantitative measurements across the physiological range of hemoglobin levels.

The wavelength range for infrared light transmission in hemoglobin monitoring applications may extend from 640 nanometers to 1000 nanometers, encompassing various absorption peaks and characteristics of hemoglobin and related blood components. The selection of the wavelength (such as that of 880 nm) for the current implementation may provide an optimal balance between measurement sensitivity and tissue penetration depth, allowing effective measurement through finger tissue while maintaining sufficient signal strength for accurate detection. Alternative wavelengths within the 640 to 1000 nanometer range may be employed in different implementations to accommodate specific measurement requirements or to provide multi-wavelength analysis capabilities for enhanced accuracy.

The optical path configuration between the transmitter and receiver may be designed to maintain consistent measurement geometry across different finger sizes and tissue characteristics. The optical components may be positioned to create a defined measurement volume within the finger placement area, ensuring that the light transmission path remains stable during measurement operations. The optical sensing system may incorporate beam shaping elements such as lenses or apertures that control the light distribution and collection characteristics, optimizing the signal-to-noise ratio and measurement repeatability. The optical alignment between transmitter and receiver components may be maintained through precise mechanical mounting structures that prevent misalignment due to mechanical stress or thermal expansion.

The photodetector response characteristics may be matched to the spectral output of the infrared transmitter to maximize measurement sensitivity and minimize noise contributions. The detector may exhibit high quantum efficiency at the wavelength (e.g., 880 nm), ensuring that a substantial portion of the received optical signal is converted into electrical current for subsequent processing. The photodetector may incorporate low-noise amplification circuitry that conditions the electrical signals for analog-to-digital conversion while preserving the hemoglobin-related signal components. The optical sensing components may be shielded from electromagnetic interference and ambient light sources through appropriate housing design and component placement strategies.

The light transmission measurement approach may provide advantages over reflectance-based optical sensing methods by creating a more direct relationship between hemoglobin concentration and measured signal levels. The transmission configuration may reduce the influence of surface tissue characteristics and skin pigmentation variations that can affect reflectance measurements, potentially improving measurement accuracy across diverse patient populations. The optical sensing components may be configured to accommodate the natural variations in finger tissue thickness and density while maintaining measurement precision through appropriate signal processing and calibration techniques.

The finger positioning and detection mechanism incorporates a tactile button system that provides confirmation of proper finger placement before measurement initiation. The tactile button may be positioned within the finger placement area to ensure that activation occurs only when the finger is properly positioned relative to the optical sensing components. The tactile button may comprise a pressure-sensitive switch element that responds to downward force applied by the user's finger during positioning. The button activation may serve as a confirmation signal that indicates adequate finger contact and proper alignment with the optical measurement path between the infrared transmitter and infrared receiver components.

The tactile button may be implemented using various switch technologies, including dome switches, membrane switches, or force-sensitive resistors that provide reliable activation characteristics under repeated use conditions. Dome switches may utilize a metal dome structure that provides tactile feedback when compressed, creating a distinct clicking sensation that confirms button activation to the user. Membrane switches may employ flexible circuit materials with conductive layers that make contact when pressure is applied, providing a low-profile button implementation that integrates seamlessly into the finger placement area surface. Force-sensitive resistors may change electrical resistance in response to applied pressure, enabling variable pressure detection and potentially providing information about finger contact quality in addition to basic activation confirmation.

The positioning of the tactile button within the finger placement area may be optimized to align with the optical measurement path while accommodating natural finger placement variations across different users. The button may be centered within the finger placement area to correspond with the typical contact point of the fingertip during normal device operation. The button surface may be slightly recessed or raised relative to the surrounding finger placement area to provide tactile guidance that helps users locate the proper finger position. The button dimensions may be sized to accommodate fingertip contact while ensuring reliable activation across different finger sizes and shapes.

The tactile button activation force may be calibrated to provide confirmation of adequate finger contact without requiring excessive pressure that could cause user discomfort or interfere with blood circulation during measurement. The activation force threshold may be set within a range that ensures reliable detection of proper finger placement while remaining accessible to users with varying finger strength or dexterity limitations. The button may provide tactile feedback through mechanical displacement or resistance changes that inform users when proper activation has been achieved. The tactile feedback characteristics may be designed to provide clear indication of button activation while maintaining user comfort throughout the measurement process.

The finger detection circuitry may monitor the tactile button activation status and communicate this information to the processing circuitry for measurement control purposes. The detection circuitry may include debouncing mechanisms that filter out spurious button activations caused by vibration or inadvertent contact, ensuring that measurement initiation occurs only when sustained button activation is detected. The circuitry may incorporate pull-up or pull-down resistors that establish defined logic states for button activation detection, providing reliable digital signals to the processing circuitry. The detection system may also include protection circuitry that prevents damage from electrostatic discharge or other electrical transients that could affect button operation.

The finger positioning mechanism may incorporate additional sensing elements that work in conjunction with the tactile button to provide comprehensive finger placement validation. Capacitive sensors may be integrated into the finger placement area to detect the presence of biological tissue and confirm that a finger is positioned over the measurement area. The capacitive sensing may provide complementary information to the tactile button activation, enabling the system to distinguish between proper finger placement and inadvertent button activation by non-biological objects. Optical sensors may monitor light transmission levels to assess finger contact quality and ensure that adequate optical coupling exists between the finger tissue and the sensing components.

The processing circuitry may implement logic algorithms that evaluate multiple finger positioning indicators to determine when measurement conditions are suitable for accurate hemoglobin concentration determination. The algorithms may require simultaneous activation of the tactile button and detection of appropriate finger presence signals before initiating the measurement sequence. The processing logic may also monitor the stability of finger positioning signals throughout the measurement process, ensuring that finger movement or contact variations do not compromise measurement accuracy. The system may provide feedback to users when finger repositioning is detected during measurement, prompting corrective action to maintain measurement quality.

The tactile button system may include visual or audible feedback mechanisms that provide confirmation of proper finger positioning and measurement readiness. Light-emitting diodes positioned near the finger placement area may illuminate when the tactile button is properly activated, providing visual confirmation that measurement can proceed. Audible feedback through speakers or buzzers may provide acoustic confirmation of button activation, particularly beneficial for users with visual limitations or in environments where visual feedback may be difficult to observe. The feedback mechanisms may be coordinated with the processing circuitry to provide sequential guidance through the finger positioning and measurement process.

The finger positioning mechanism may accommodate variations in finger anatomy and positioning preferences through adjustable or adaptive features. The tactile button sensitivity may be programmable to accommodate users with different finger pressure capabilities or preferences. The finger placement area may include textured surfaces or guide features that help users locate the optimal finger position relative to the tactile button and optical sensing components. The positioning mechanism may also account for natural finger curvature and contact patterns, ensuring that the tactile button activation occurs reliably across different finger positioning approaches.

The detection circuitry may implement diagnostic capabilities that monitor tactile button functionality and detect potential malfunctions or degradation over time. The diagnostic system may perform periodic button response tests during device initialization or maintenance cycles, verifying that activation forces and electrical responses remain within acceptable ranges. The circuitry may also monitor button activation patterns during normal use to identify potential issues such as mechanical wear, contamination, or electrical contact problems. The diagnostic information may be stored in device memory and made available to service personnel for preventive maintenance or troubleshooting purposes.

The processing circuitry comprises a microcontroller unit and associated signal processing components that coordinate measurement operations and perform hemoglobin concentration calculations based on optical sensor data. The microcontroller may be implemented using an ATMEGA328P-20MU or similar microprocessor device that provides sufficient computational capability for real-time signal processing and system control functions. The microcontroller may operate at clock frequencies of 16 MHz or higher to ensure adequate processing speed for data acquisition and calculation operations. The processing circuitry may be coupled to both the infrared receiver and the tactile button through appropriate interface circuits that condition signals for digital processing.

The microcontroller may be configured to monitor the tactile button activation status continuously and initiate measurement sequences only when proper finger positioning is confirmed through button activation. The processing circuitry may implement state machine logic that transitions between idle, measurement preparation, data acquisition, and result calculation states based on tactile button status and other system conditions. The microcontroller may include multiple input/output pins that interface with various system components, including analog input pins for sensor data acquisition and digital input/output pins for tactile button monitoring and system control functions. The processing circuitry may incorporate interrupt handling capabilities that enable responsive system operation and efficient power management during measurement operations.

The signal processing electronics may be configured to convert analog signals from the photodetector to digital values suitable for computational analysis and hemoglobin concentration determination. The analog-to-digital conversion process may utilize integrated ADC circuits within the microcontroller or external ADC components that provide higher resolution or sampling rates than internal converters. The ADC circuits may operate at 10-bit, 12-bit, or 16-bit resolution to capture the subtle variations in optical signal levels that correspond to hemoglobin concentration differences. The sampling rate of the ADC may be configured to provide adequate temporal resolution for capturing physiological variations while avoiding aliasing effects that could compromise measurement accuracy.

The processing circuitry may be configured to acquire multiple sensor readings from the infrared receiver when proper finger positioning is confirmed through tactile button activation. In some cases, the microcontroller may be configured to acquire 20 sensor readings from the photodetector with a 0.5-second delay between each reading to ensure measurement stability and reduce the influence of physiological artifacts such as pulse variations or breathing-related tissue movement. The multiple reading acquisition process may span a total measurement time of approximately 10 seconds, providing sufficient data for statistical analysis while maintaining reasonable measurement duration for user convenience. The timing between sensor readings may be controlled through software timers or hardware timing circuits that ensure consistent sampling intervals regardless of other processing activities.

The sensor reading acquisition process may incorporate signal conditioning algorithms that filter noise and artifacts from the raw optical sensor data before storage and analysis. The signal conditioning may include digital filtering techniques such as low-pass filtering to remove high-frequency noise components or band-pass filtering to isolate the frequency range associated with hemoglobin-related signal variations. The processing circuitry may implement averaging filters or median filters that reduce the impact of outlier readings caused by motion artifacts or electrical interference. The filtered sensor readings may be stored in temporary memory locations within the microcontroller for subsequent averaging and calculation operations.

The processing circuitry may be configured to calculate an average value from the multiple sensor readings to produce a single representative value for hemoglobin calculation. The averaging calculation may involve summing all acquired sensor readings and dividing by the number of readings to produce an arithmetic mean value. In some cases, the averaging process may incorporate weighted averaging techniques that assign different importance levels to individual readings based on signal quality metrics or temporal position within the measurement sequence. The calculated average value may undergo additional processing such as offset correction or gain normalization to account for system variations or calibration factors that affect measurement accuracy.

The hemoglobin concentration determination may be performed using a predetermined algorithm that processes the average sensor value through mathematical relationships derived from calibration studies and theoretical models. The predetermined algorithm may comprise a compartmental model approach that accounts for the complex optical properties of biological tissue and the distribution of hemoglobin within blood vessels. The compartmental model algorithm may incorporate multiple mathematical compartments that represent different tissue layers, blood vessel sizes, and hemoglobin distribution patterns encountered during optical transmission measurements. The algorithm may utilize empirically derived coefficients and correction factors that optimize measurement accuracy across diverse patient populations and physiological conditions.

The processing circuitry may implement algorithm selection logic that chooses between different calculation formulas based on the measured signal characteristics and average sensor values. In some cases, the predetermined algorithm may comprise selecting between a first calculation formula and a second calculation formula based on whether the average value is less than 1017 or greater than or equal to 1017. The threshold value of 1017 may correspond to a specific signal level that indicates different optical transmission regimes or tissue characteristics that benefit from different mathematical approaches. The first calculation formula may be optimized for lower signal levels that correspond to higher hemoglobin concentrations or thicker tissue samples, while the second calculation formula may be optimized for higher signal levels associated with lower hemoglobin concentrations or thinner tissue samples.

The compartmental model algorithm may incorporate multiple mathematical equations that describe the relationship between optical transmission characteristics and hemoglobin concentration based on Beer-Lambert law principles and tissue scattering effects. The model may account for the wavelength-dependent absorption characteristics of hemoglobin, the scattering properties of tissue components, and the geometric factors associated with the optical measurement configuration. The compartmental approach may divide the measurement volume into discrete regions with different optical properties, enabling more accurate modeling of the complex light propagation patterns that occur during transmission measurements through biological tissue. The algorithm may utilize iterative calculation methods or lookup table approaches to solve the compartmental equations efficiently within the computational constraints of the microcontroller.

The data storage capabilities of the processing circuitry may incorporate multiple memory types that serve different functions within the hemoglobin monitoring system. Flash memory may provide non-volatile storage for program code, calibration parameters, and firmware components that define the operational behavior of the device. The flash memory capacity may range from 32 kilobytes to several megabytes, depending on the complexity of the processing algorithms and the amount of calibration data that requires storage. SRAM may provide volatile memory for temporary data storage during program execution, including variables, arrays, and intermediate calculation results generated during the measurement process. The SRAM capacity may be sized to accommodate the data processing requirements of the signal processing algorithms while maintaining efficient memory utilization.

EEPROM may provide electrically erasable programmable read-only memory for long-term storage of calibration data, device configuration parameters, and measurement history information that should be retained after power loss. The EEPROM may store factory calibration coefficients that are determined during manufacturing testing and quality assurance procedures. The memory may also store user-specific calibration adjustments or device configuration settings that personalize the measurement algorithms for individual users or specific measurement applications. The EEPROM capacity may range from 1 kilobyte to several kilobytes, providing sufficient storage for calibration parameters and limited measurement history while maintaining cost-effective memory utilization.

The processing circuitry may implement memory management algorithms that optimize the utilization of available memory resources while ensuring reliable data storage and retrieval operations. The memory management may include wear leveling techniques for EEPROM that distribute write operations across different memory locations to extend the operational lifetime of the memory components. The system may implement data integrity checking mechanisms such as checksums or error correction codes that detect and correct memory errors that could affect measurement accuracy or system reliability. The memory management may also include data compression techniques that reduce the storage requirements for measurement history or calibration data while maintaining adequate precision for system operation.

The signal processing algorithms may incorporate real-time processing capabilities that enable immediate calculation and display of hemoglobin concentration results following data acquisition completion. The real-time processing may utilize optimized mathematical routines and efficient memory access patterns that minimize calculation time while maintaining measurement precision. The processing circuitry may implement parallel processing techniques that overlap data acquisition and calculation operations to reduce overall measurement time. The algorithms may also incorporate predictive processing methods that begin preliminary calculations during the data acquisition phase, enabling faster result presentation to users upon completion of the measurement sequence.

The display component may comprise various digital display technologies configured to present hemoglobin concentration measurements and system status information to users during device operation. The display may be implemented using liquid crystal display (LCD) technology that provides clear numerical and textual presentation of information with low power consumption characteristics suitable for battery-operated medical devices. LCD displays may utilize thin-film transistor (TFT) technology that enables active matrix control of individual pixels, providing enhanced contrast ratios and viewing angle characteristics compared to passive matrix display technologies. The TFT LCD configuration may support color display capabilities that enable the presentation of measurement results in different colors to indicate normal, warning, or error conditions based on hemoglobin concentration values.

Alternatively, the display may be implemented using organic light-emitting diode (OLED) technology that provides self-illuminating pixels without requiring separate backlighting components. OLED displays may offer advantages in terms of contrast ratio, response time, and power consumption for certain display content patterns, particularly when displaying high-contrast information such as numerical measurement results against dark backgrounds. The OLED technology may enable thinner display modules compared to LCD implementations, potentially contributing to reduced device thickness and improved portability characteristics. The OLED displays may also provide wider viewing angles and better visibility under various ambient lighting conditions encountered in clinical and home-use environments.

The display specifications may include a 1.77-inch diagonal screen size that provides adequate viewing area for presenting hemoglobin concentration values, measurement units, and status information while maintaining compact device dimensions suitable for portable operation. The display resolution may be configured as 128 pixels horizontally by 160 pixels vertically, providing sufficient pixel density for clear presentation of numerical values and textual information. The RGB color capability may enable the display to present information in multiple colors, with red, green, and blue color channels that can be combined to produce various color combinations for different types of information presentation. The pixel arrangement may utilize standard RGB stripe patterns or alternative pixel geometries that optimize color reproduction and text clarity for medical measurement applications.

The display may be coupled to the processing circuitry through serial peripheral interface (SPI) communication protocols that enable efficient data transfer between the microcontroller and display controller components. The SPI interface may provide high-speed data transmission capabilities that support real-time updating of display content during measurement operations and result presentation. The display controller may incorporate frame buffer memory that stores pixel data for the entire display area, enabling smooth transitions between different display screens and reducing the processing burden on the main microcontroller during display updates. The communication interface may include control signals for display initialization, power management, and synchronization of data transfers between the processing circuitry and display components.

The display may be configured to present hemoglobin concentration values in standard medical units such as grams per deciliter (g/dL) with appropriate decimal precision to provide clinically relevant measurement resolution. The numerical display format may include large, easily readable fonts that accommodate users with varying visual capabilities and enable clear reading under different lighting conditions. The measurement results may be presented with accompanying unit labels and measurement timestamp information that provides context for the displayed values. The display layout may incorporate graphical elements such as progress bars during measurement acquisition or battery status indicators that inform users of device operational status without interfering with the primary measurement result presentation.

The display may be configured to present error messages when calculated hemoglobin concentration values fall outside predetermined physiological ranges or when measurement quality issues are detected during the acquisition process. The error message presentation may utilize distinct visual formatting such as different text colors, flashing indicators, or warning symbols that clearly distinguish error conditions from normal measurement results. The error messages may include specific textual descriptions such as “Finger Placement Error” or “Measurement Out of Range” that provide users with clear information about the nature of the detected problem. The error message display may remain visible for predetermined time periods to ensure that users have adequate opportunity to read and understand the error condition before the display returns to normal operation modes.

The error message system may be configured to provide specific guidance for corrective actions when measurement problems are detected. Error messages indicating improper finger placement may prompt users to reposition their finger in the finger placement area and attempt the measurement process again. The display may present sequential error messages that guide users through troubleshooting procedures, such as checking finger contact with the tactile button or ensuring adequate finger coverage of the optical sensing area. The error message presentation may include countdown timers or progress indicators that inform users of the time remaining before automatic return to measurement-ready status or the initiation of retry procedures.

The display brightness and contrast settings may be adjustable to accommodate different ambient lighting conditions and user preferences encountered during device operation. The brightness control may be implemented through pulse-width modulation (PWM) techniques that adjust the effective illumination intensity of LCD backlighting or OLED pixel drive currents. The contrast adjustment may modify the voltage levels applied to display elements to optimize the visual distinction between different display elements and background areas. The display settings may be stored in non-volatile memory to maintain user preferences across power cycles and device usage sessions.

The user interface elements may incorporate visual indicators such as light-emitting diodes (LEDs) positioned on the device housing to provide status information that complements the main display functionality. The LED indicators may include power status lights that indicate when the device is active and ready for measurement operations. Measurement progress indicators may utilize different LED colors or flashing patterns to communicate the current stage of the measurement process, such as finger positioning, data acquisition, or result calculation phases. The LED indicators may be positioned to provide clear visibility during normal device handling and operation, with appropriate light intensity levels that provide adequate visibility without causing user discomfort or excessive power consumption.

The display and user interface components may implement power management features that optimize battery life while maintaining adequate functionality for measurement operations. The display may incorporate automatic dimming or sleep modes that reduce power consumption during periods of inactivity while maintaining the ability to quickly resume full brightness when user interaction is detected. The power management may include ambient light sensing capabilities that automatically adjust display brightness based on surrounding lighting conditions, optimizing visibility while minimizing unnecessary power consumption. The interface components may also implement selective activation of display elements, enabling the presentation of minimal status information during standby modes while reserving full display functionality for active measurement periods.

The validation mechanisms for hemoglobin concentration readings may incorporate multiple levels of quality assessment to ensure measurement reliability and clinical relevance. The processing circuitry may be configured to validate calculated hemoglobin concentration values by checking whether the concentration falls within a predetermined range of 1 to 17.5 grams per deciliter, which encompasses the physiological range of hemoglobin concentrations found in both healthy individuals and patients with various hematological conditions. The validation process may compare calculated values against established medical reference ranges that account for age, gender, and physiological variations in hemoglobin levels across different patient populations. The predetermined range boundaries may be based on clinical literature and regulatory guidelines that define acceptable hemoglobin concentration limits for diagnostic applications.

The range checking algorithms may implement multiple threshold comparisons to categorize measurement results into valid, borderline, or invalid categories based on the calculated hemoglobin concentration values. Values falling below 1 gram per deciliter may indicate severe anemia, measurement errors, or device malfunction conditions that require further evaluation or corrective action. Concentrations exceeding 17.5 grams per deciliter may suggest polycythemia, dehydration, or measurement artifacts that could compromise the clinical utility of the results. The validation system may incorporate additional sub-ranges within the overall 1 to 17.5 g/dL range that trigger different types of user notifications or measurement quality indicators based on the clinical significance of specific concentration levels.

The error message generation system may provide specific textual feedback when calculated hemoglobin concentrations fall outside the predetermined physiological range. The processing circuitry may be configured to display error messages such as “Measurement Out of Range” or “Invalid Reading Detected” when validation checks identify concentration values that exceed the acceptable limits. The error messages may include numerical information that indicates the calculated value and the acceptable range boundaries, enabling users to assess the magnitude of the measurement deviation. The message presentation may utilize distinct visual formatting such as different text colors, warning symbols, or flashing indicators that clearly distinguish error conditions from normal measurement results.

The error handling mechanisms may incorporate automatic retry capabilities that prompt users to reposition their finger and attempt measurement again when validation failures are detected. The system may track the number of consecutive measurement attempts and provide escalating guidance or recommendations for device maintenance when repeated validation failures occur. The error handling logic may distinguish between different types of validation failures, such as those caused by improper finger placement versus those indicating potential device malfunction or calibration drift. The processing circuitry may implement timeout mechanisms that prevent indefinite retry attempts while providing users with clear guidance on when to seek technical support or device servicing.

The finger placement guidance system may provide specific instructions to users when measurement validation indicates problems related to improper finger positioning or inadequate optical coupling. Error messages indicating finger placement issues may include detailed instructions such as “Press tactile button firmly” or “Ensure complete finger coverage of sensing area” that guide users toward proper measurement technique. The guidance system may provide sequential prompts that walk users through the finger positioning process step by step, including instructions for finger alignment, pressure application, and contact duration requirements. The error handling may incorporate visual or audible feedback mechanisms that provide real-time guidance during finger repositioning attempts.

The measurement quality assessment algorithms may analyze multiple signal characteristics beyond basic range checking to identify potential sources of measurement error or uncertainty. The validation system may evaluate signal stability during the data acquisition period, checking for excessive variations that could indicate finger movement or inadequate contact pressure. The algorithms may assess signal-to-noise ratios to determine whether acquired data meets minimum quality standards for reliable hemoglobin calculation. The quality assessment may include analysis of measurement repeatability by comparing current results with recent measurement history when available, identifying potential inconsistencies that warrant additional validation or user guidance.

The error classification system may categorize different types of measurement problems to provide targeted corrective guidance and improve measurement success rates. Systematic errors related to finger placement may trigger specific repositioning instructions and tactile button activation reminders. Random errors associated with signal noise or interference may prompt users to ensure stable finger contact and minimize movement during measurement acquisition. The classification system may maintain error statistics that track the frequency and types of measurement problems encountered, potentially identifying patterns that indicate device maintenance needs or user training opportunities.

The validation system may implement progressive error handling that provides increasingly detailed guidance as measurement attempts continue to fail validation checks. Initial error messages may provide basic repositioning instructions, while subsequent failures may trigger more comprehensive troubleshooting guidance that addresses potential environmental factors or device operation issues. The progressive approach may include recommendations for cleaning the finger placement area, checking battery status, or ensuring adequate ambient lighting conditions that could affect measurement quality. The system may also provide contact information or references to user documentation when multiple validation failures suggest the need for additional technical support.

The measurement confidence indicators may provide users with information about the reliability and quality of validated measurement results even when values fall within the acceptable range. The confidence assessment may consider factors such as signal strength, measurement stability, and consistency with previous readings to generate quality scores or reliability ratings. The indicators may be presented through graphical elements such as progress bars, star ratings, or color-coded symbols that communicate measurement confidence levels without requiring detailed technical interpretation. The confidence information may help users and healthcare providers assess the clinical utility of measurement results and determine when additional measurements or confirmatory testing may be appropriate.

The power management system of the non-invasive hemoglobin monitoring device comprises a rechargeable battery power supply and associated electrical circuits that provide stable power distribution throughout the device components while maintaining operational safety and extended battery life. The rechargeable battery power supply may comprise lithium-ion or lithium-polymer battery cells that provide energy storage capacity suitable for multiple measurement sessions between charging cycles. The battery cells may be configured with a nominal voltage of 3.7 volts and capacity ratings ranging from 250 milliampere-hours to several ampere-hours, depending on the power consumption requirements of the device components and the desired operational duration between charging events. The battery selection may consider factors including energy density, cycle life, safety characteristics, and temperature stability to ensure reliable operation across various environmental conditions encountered in medical device applications.

The charging circuit may incorporate protection mechanisms against overcharging and overheating conditions that could damage the battery cells or create safety hazards during charging operations. The charging protection circuitry may utilize integrated circuit components such as TP4056 charging controller chips that provide constant current and constant voltage charging profiles optimized for lithium-ion battery chemistry. The TP4056 charging controller may regulate the charging current during the initial charging phase and transition to voltage regulation mode as the battery approaches full charge capacity. The charging circuit may also incorporate FS8205 dual MOSFET protection devices that provide overcurrent protection and reverse polarity protection during charging and discharging operations. The DW01A battery protection integrated circuit may monitor battery voltage levels and disconnect the battery from the charging circuit when overvoltage or undervoltage conditions are detected, preventing damage to the battery cells and associated circuitry.

The charging interface may utilize USB-C connector technology that provides convenient connection to standard charging adapters and computer USB ports for battery recharging operations. The USB-C connector may support various charging protocols and current levels, enabling compatibility with different charging sources while maintaining safe charging parameters. The charging circuit may include status indication LEDs that provide visual feedback about charging progress and battery condition during charging operations. The status indicators may utilize different colors or flashing patterns to communicate charging states such as charging in progress, charging complete, or charging fault conditions. The charging circuit may also incorporate thermal monitoring capabilities that reduce charging current or suspend charging operations when elevated temperatures are detected, preventing thermal damage to battery cells or surrounding components.

The voltage regulation system may include boost-out circuitry that amplifies the battery voltage from the nominal 3.7 volts to a constant 5 volts to maintain voltage uniformity across device components that require higher operating voltages. The boost-out circuit may utilize MT3608 step-up converter integrated circuits that provide efficient voltage conversion with minimal power loss during the voltage amplification process. The MT3608 boost converter may incorporate switching regulator topology that uses inductors and capacitors to store and transfer energy during the voltage conversion process, achieving conversion efficiencies exceeding 90 percent under typical operating conditions. The boost circuit may include feedback control mechanisms that maintain stable 5-volt output regardless of battery voltage variations that occur as the battery discharges during normal device operation.

The boost-out circuit may incorporate various passive components that support the voltage conversion process and ensure stable operation across different load conditions. Inductors with values such as 22 microhenries may store energy during the switching cycles of the boost converter, while capacitors with values such as 22 microfarads may filter the output voltage and reduce ripple components that could interfere with sensitive electronic circuits. Schottky diodes such as SS34 devices may provide low forward voltage drop characteristics that improve conversion efficiency by minimizing power losses during the switching operations. Resistor networks may establish feedback voltage levels that control the output voltage regulation and ensure accurate voltage levels across varying load and temperature conditions.

The switching circuit may provide on/off control functionality through push button activation that enables users to power the device on and off as needed for measurement operations. The switching circuit may utilize MOSFET transistor devices such as MMBT2222A and A03407A components that provide low resistance switching characteristics with minimal power consumption during both on and off states. The MOSFET switching elements may be configured in circuit topologies that provide complete disconnection of battery power from device circuits during off states, preventing parasitic current draw that could discharge the battery during storage periods. The switching circuit may incorporate debouncing mechanisms that filter spurious button activations and ensure reliable power state transitions when users activate the power button.

The power indication system may include LED indicators that provide visual feedback about device power status and operational readiness. The power indication LED may utilize red light emission to provide clear visibility under various ambient lighting conditions encountered during device operation. The LED may be positioned on the device housing exterior and connected to the power management circuitry through current limiting resistors that establish appropriate brightness levels while preventing excessive current draw from the battery supply. The power indication circuit may include switching logic that activates the LED when device power is applied and maintains illumination throughout active operational periods. The LED indication system may also provide different illumination patterns such as steady illumination during normal operation or flashing patterns during low battery conditions or charging states.

The battery level indication circuit may utilize voltage divider configurations that monitor battery voltage levels and provide information about remaining battery capacity to users through display presentations. The voltage divider circuit may comprise precision resistors with values such as 10 kilohms that create voltage reference levels proportional to the battery voltage, enabling the processing circuitry to determine battery charge status through analog-to-digital conversion measurements. The battery level monitoring may account for the voltage discharge characteristics of lithium-ion batteries, correlating measured voltage levels with remaining capacity percentages based on empirical discharge curves and temperature compensation factors. The battery level information may be presented on the device display as percentage values or graphical battery icons that provide intuitive indication of remaining operational time.

The power management system may implement power-saving operational modes that extend battery life during periods of reduced activity or standby conditions. The power management may include automatic shutdown timers that power down non-essential circuits after predetermined periods of inactivity, while maintaining minimal power consumption for rapid wake-up when user interaction is detected. The power-saving modes may selectively disable high-power components such as display backlighting or optical transmitters while preserving low-power circuits that monitor user input and system status. The power management algorithms may implement dynamic voltage scaling that reduces supply voltages to digital circuits during low-performance operational periods, reducing power consumption while maintaining adequate functionality for system monitoring and user interface operations.

The power distribution architecture may incorporate multiple voltage rails that supply different voltage levels to various device components based on their specific power requirements. The power distribution may include 3.3-volt rails for digital processing circuits, 5-volt rails for optical components and display systems, and specialized voltage levels for analog circuits that require precise voltage references. The voltage rail generation may utilize linear regulators or switching regulators that provide stable voltage outputs with low noise characteristics suitable for sensitive analog and digital circuits. The power distribution system may include decoupling capacitors and filtering components that reduce electrical noise and voltage fluctuations that could affect measurement accuracy or system stability.

The power management system may incorporate diagnostic capabilities that monitor power system performance and detect potential issues such as battery degradation, charging circuit malfunctions, or voltage regulation problems. The diagnostic system may track battery charging and discharging cycles to estimate battery health and remaining service life, providing maintenance recommendations when battery replacement becomes advisable. The power monitoring may include measurement of current consumption patterns that identify abnormal power draw conditions that could indicate component malfunctions or system faults. The diagnostic information may be stored in non-volatile memory and made available to service personnel for troubleshooting and preventive maintenance purposes, enabling proactive system maintenance that prevents unexpected power-related failures during clinical use.

The programming interface capabilities of the non-invasive hemoglobin monitoring device may incorporate external connection systems that enable firmware updates, calibration parameter modifications, and configuration changes through standardized communication protocols. The programming interface may comprise dedicated programming circuits that provide access to the microcontroller programming functions without requiring device disassembly or component removal. The programming circuits may utilize FT232RL FTDI integrated circuit components that convert USB communication signals to serial communication protocols compatible with microcontroller programming interfaces. The FT232RL FTDI chip may provide bidirectional data transfer capabilities that support both programming operations and diagnostic data retrieval from the device memory systems.

The FT232RL FTDI programming circuit may incorporate USB interface connections that enable direct connection to computer systems for firmware development, testing, and field update operations. The FTDI interface may support various baud rates and communication protocols that accommodate different programming software packages and development environments used for microcontroller programming. The programming circuit may include signal conditioning components such as resistors and capacitors that ensure reliable data transmission during programming operations while protecting the microcontroller from electrical transients or voltage spikes that could occur during connection and disconnection procedures. The programming interface may also incorporate isolation mechanisms that prevent interference between programming signals and normal device operation circuits.

The connector technology for programming and data transfer operations may utilize USB-C connector implementations that provide enhanced data transfer capabilities compared to previous connector technologies. The transition from Micro USB to USB-C connector types may provide improved mechanical durability, reversible connection orientation, and higher current carrying capacity for charging operations. USB-C connectors may support multiple communication protocols and power delivery standards that enable compatibility with various charging adapters and computer interfaces encountered in clinical and development environments. The USB-C interface may incorporate shielding and grounding features that reduce electromagnetic interference during data transfer operations while maintaining signal integrity across different cable lengths and connection configurations.

The programming circuit may support in-system programming (ISP) methodologies that enable firmware updates without removing the microcontroller from the device circuitry. The ISP capability may utilize dedicated programming pins on the microcontroller that remain accessible through the programming interface during normal device operation. The programming interface may include protection mechanisms such as pull-up resistors and bypass capacitors that maintain stable signal levels during programming operations while preventing inadvertent activation of programming modes during normal device use. The ISP functionality may enable field updates of device firmware to incorporate new features, calibration improvements, or regulatory compliance modifications without requiring device return to manufacturing facilities.

The wireless connectivity features may incorporate Bluetooth communication capabilities that enable data synchronization with smartphones, tablet computers, and other mobile devices commonly used in healthcare environments. The Bluetooth implementation may utilize low-energy protocols that minimize power consumption while maintaining reliable data transmission over typical operating distances encountered during patient monitoring applications. The Bluetooth interface may support various data formats and communication protocols that enable integration with health monitoring applications and electronic health record systems. The wireless communication may include authentication and encryption mechanisms that protect patient data during transmission and ensure compliance with healthcare data security regulations.

The Wi-Fi connectivity options may provide network-based communication capabilities that enable data transfer to cloud-based health platforms and remote monitoring systems. The Wi-Fi implementation may support standard wireless networking protocols that enable connection to hospital networks, clinic wireless systems, and home internet connections. The wireless interface may incorporate automatic network discovery and connection management features that simplify device setup and reduce technical complexity for end users. The Wi-Fi connectivity may enable real-time data transmission during measurement operations or batch data transfer during scheduled synchronization periods, depending on network availability and user preferences.

The data synchronization capabilities may incorporate automatic and manual synchronization modes that accommodate different usage patterns and connectivity preferences. The automatic synchronization may activate when wireless connections are detected and available, transferring measurement data and device status information to designated health platforms or mobile applications. The manual synchronization may enable users to initiate data transfer operations at convenient times or when specific network connections are available. The synchronization process may include data compression and error checking mechanisms that ensure reliable data transfer while minimizing bandwidth requirements and transmission time.

The programming and connectivity systems may incorporate security features that prevent unauthorized access to device firmware and protect patient data during transmission operations. The security implementation may include encrypted communication protocols that scramble data during wireless transmission to prevent interception by unauthorized parties. The programming interface may incorporate access control mechanisms that require authentication credentials or security keys before enabling firmware modification operations. The security system may also include audit logging capabilities that record programming and data access activities for compliance monitoring and troubleshooting purposes.

The connector interface may incorporate environmental protection features that maintain reliable electrical connections while preventing contamination from cleaning agents and sterilization procedures commonly used in healthcare environments. The USB-C connector may include sealing mechanisms or protective covers that prevent moisture and debris ingress when the connector is not in use. The connector design may utilize corrosion-resistant materials and plating that maintain electrical conductivity and mechanical integrity under repeated connection cycles and exposure to cleaning chemicals. The interface may also incorporate strain relief features that prevent cable damage and connection failure due to mechanical stress during normal handling and use.

The programming circuit may include diagnostic capabilities that monitor communication interface performance and detect potential connectivity issues that could affect programming or data transfer operations. The diagnostic system may perform periodic communication tests that verify the integrity of programming connections and wireless communication links. The diagnostics may include signal quality monitoring that assesses data transmission reliability and identifies potential sources of communication errors or interference. The diagnostic information may be stored in device memory and made available through the programming interface for troubleshooting and system maintenance purposes, enabling proactive identification and resolution of connectivity issues before the connectivity issues affect device functionality or data synchronization operations.

The method of operation for the non-invasive hemoglobin monitoring device encompasses a comprehensive sequence of operational steps that guide users through the complete measurement process from device activation through final result presentation. The operational method may begin with device initialization procedures that verify system functionality and prepare the optical sensing components for measurement operations. The initialization sequence may include power-on self-test routines that check the operational status of the infrared transmitter, infrared receiver, processing circuitry, and display components to ensure proper functionality before measurement attempts. The self-test procedures may verify that optical components produce appropriate signal levels, processing circuits respond correctly to test inputs, and display systems present information clearly and accurately.

The device activation process may involve user interaction with power control mechanisms that transition the system from standby or off states to active measurement-ready conditions. The activation sequence may include battery status verification that confirms adequate power levels for completing measurement operations without interruption due to low battery conditions. The system may perform calibration checks that verify the accuracy of optical sensing components and processing algorithms against stored reference values or self-calibration procedures. The activation process may also include environmental condition assessments that evaluate ambient light levels, temperature conditions, and other factors that could influence measurement accuracy or system performance.

The finger placement phase of the operational method may commence with user guidance through visual or audible prompts that direct proper finger positioning within the designated finger placement area of the device housing. The guidance system may provide sequential instructions that help users locate the optimal finger position relative to the optical sensing components and tactile feedback mechanisms. The finger placement process may involve positioning the finger to cover the optical measurement path between the infrared transmitter and infrared receiver while ensuring adequate contact with the tactile button positioned within the finger placement area. The positioning guidance may include instructions for finger orientation, contact pressure, and alignment that optimize optical coupling and measurement accuracy.

The finger positioning confirmation may utilize tactile button activation as a verification mechanism that indicates proper finger placement before measurement initiation. The tactile button activation process may require users to apply sufficient downward pressure to compress the button mechanism and generate an electrical signal that confirms adequate finger contact. The button activation may serve as a gating mechanism that prevents measurement attempts when finger positioning is inadequate or when optical alignment conditions are suboptimal. The confirmation process may include feedback mechanisms such as visual indicators or audible signals that inform users when proper finger positioning has been achieved and measurement operations can proceed.

The optical measurement sequence may begin with infrared transmitter activation that generates light emission at the designated wavelength (such as that of 880 nanometers) through the finger placement area. The light emission process may involve current regulation circuits that establish stable optical output levels while minimizing power consumption and thermal generation during measurement operations. The infrared light transmission may pass through finger tissue positioned within the optical measurement path, with hemoglobin and other tissue components absorbing portions of the transmitted light according to their spectral absorption characteristics. The light transmission process may continue throughout the data acquisition period to provide consistent optical conditions for measurement stability and accuracy.

The data acquisition phase may involve systematic collection of multiple sensor readings from the infrared receiver over predetermined time intervals to ensure measurement stability and reduce the influence of physiological variations. The acquisition process may collect sensor readings at regular intervals with specified delays between individual measurements to account for natural variations in blood flow, tissue movement, and other physiological factors that could affect optical transmission characteristics. The data collection may span sufficient time periods to capture representative samples of the optical transmission conditions while maintaining reasonable measurement duration for user convenience and comfort.

The signal processing operations may commence during or immediately following the data acquisition phase to convert raw optical sensor data into processed values suitable for hemoglobin concentration calculations. The signal processing may include analog-to-digital conversion procedures that transform electrical signals from the photodetector into digital values with appropriate resolution and accuracy for computational analysis. The processing operations may incorporate filtering algorithms that remove noise components and artifacts from the acquired sensor data while preserving the hemoglobin-related signal characteristics. The signal conditioning may also include gain adjustments and offset corrections that normalize the sensor data to account for system variations and calibration factors.

The averaging calculations may process the multiple acquired sensor readings to produce single representative values that reduce the impact of random variations and measurement noise on the final hemoglobin concentration determination. The averaging process may utilize arithmetic mean calculations that sum all acquired readings and divide by the number of samples to produce statistically representative values. The averaging operations may also incorporate weighted averaging techniques that assign different importance levels to individual readings based on signal quality metrics or temporal position within the measurement sequence. The calculated average values may undergo additional processing such as outlier rejection or statistical filtering to further improve measurement accuracy and reliability.

The hemoglobin concentration calculation may utilize predetermined algorithms that convert the processed sensor data into clinically relevant hemoglobin concentration values expressed in standard medical units. The calculation algorithms may implement mathematical relationships derived from calibration studies and theoretical models that correlate optical transmission characteristics with hemoglobin concentration levels. The algorithmic processing may include compartmental model approaches that account for the complex optical properties of biological tissue and the distribution of hemoglobin within blood vessels and tissue structures. The calculation process may also incorporate correction factors and calibration coefficients that optimize measurement accuracy across diverse patient populations and physiological conditions.

The algorithm selection process may evaluate the characteristics of the processed sensor data to determine the most appropriate calculation method for the specific measurement conditions encountered. The selection logic may compare average sensor values against predetermined thresholds that indicate different optical transmission regimes or tissue characteristics that benefit from different mathematical approaches. The algorithm selection may choose between multiple calculation formulas that are optimized for different signal level ranges or tissue types to maximize measurement accuracy across varying measurement conditions. The selection process may also consider historical measurement data or user-specific calibration parameters when determining the optimal calculation approach.

The validation procedures may assess the calculated hemoglobin concentration values against predetermined physiological ranges and quality criteria to ensure measurement reliability and clinical relevance. The validation process may compare calculated concentrations against established medical reference ranges that encompass normal and pathological hemoglobin levels found in clinical practice. The validation algorithms may also evaluate measurement consistency by comparing current results with recent measurement history when available, identifying potential inconsistencies that warrant additional validation or user guidance. The validation process may incorporate signal quality assessments that analyze the characteristics of the acquired sensor data to determine measurement confidence levels and reliability indicators.

The error detection and handling procedures may identify measurement problems and provide appropriate user guidance when validation checks indicate suboptimal measurement conditions or results. The error detection may recognize various types of measurement issues including improper finger placement, inadequate optical coupling, signal quality problems, or calculated values that fall outside acceptable physiological ranges. The error handling system may generate specific error messages that inform users about the nature of detected problems and provide guidance for corrective actions. The error response may include prompts for finger repositioning, measurement retry procedures, or recommendations for device maintenance when systematic problems are identified.

The result presentation phase may display the validated hemoglobin concentration values on the device screen along with accompanying information such as measurement units, timestamp data, and quality indicators. The presentation format may utilize clear numerical displays with appropriate decimal precision and unit labels that provide clinically relevant information in easily readable formats. The result display may also include graphical elements or color coding that indicates whether measured values fall within normal ranges or warrant clinical attention. The presentation system may maintain result displays for predetermined time periods to ensure adequate user review time before returning to measurement-ready states or powering down to conserve battery life.

The data storage operations may record measurement results and associated metadata in device memory systems for future reference and trend analysis. The storage process may include timestamp information, measurement quality indicators, and user identification data when applicable to create comprehensive measurement records. The stored data may be organized in formats that facilitate retrieval and analysis while maintaining data integrity and security. The storage system may also implement data management procedures that prevent memory overflow conditions and maintain adequate storage capacity for ongoing measurement operations.

The measurement completion procedures may include system cleanup operations that prepare the device for subsequent measurements or standby modes. The completion process may involve optical component deactivation to conserve battery power and reduce thermal generation when measurements are not in progress. The system may also perform diagnostic checks that assess component performance and identify potential maintenance needs based on measurement frequency and system usage patterns. The completion procedures may include user prompts that indicate measurement completion and provide options for additional measurements or device shutdown based on user preferences and operational requirements.

The system integration of the non-invasive hemoglobin monitoring device encompasses the coordinated operation of multiple subsystems that work together to provide accurate and reliable hemoglobin concentration measurements through optical sensing technology. The integration architecture may utilize a hierarchical control approach where the main processing circuitry serves as the central coordination hub for all device subsystems, managing data flow, timing sequences, and operational states throughout the measurement process. The processing circuitry may communicate with various subsystems through standardized electrical interfaces and communication protocols that ensure reliable data transfer and synchronized operation across different functional components. The system coordination may implement real-time scheduling algorithms that manage the timing of optical sensing operations, data acquisition sequences, and display updates to provide seamless user experience while maintaining measurement accuracy and system stability.

The optical sensing subsystem may interface with the processing circuitry through analog signal conditioning circuits that prepare optical sensor signals for digital processing and analysis. The signal conditioning interface may include amplification stages that boost weak photodetector signals to levels suitable for analog-to-digital conversion while maintaining signal integrity and minimizing noise contributions. The conditioning circuits may incorporate filtering components that remove unwanted frequency components and electrical interference from the optical signals before digital conversion. The interface between optical sensing and processing subsystems may utilize multiple signal paths that carry both measurement data and control signals, enabling the processing circuitry to monitor optical component status and adjust operating parameters based on measurement conditions and system requirements.

The measurement workflow coordination may begin with system initialization procedures that establish communication links between all subsystems and verify operational readiness before measurement operations commence. The initialization sequence may involve systematic testing of optical transmitter output levels, photodetector response characteristics, and signal processing pathway integrity to ensure that all components function within acceptable parameters. The processing circuitry may coordinate the activation sequence of different subsystems, powering up optical components only when measurement operations are imminent to conserve battery power and reduce thermal generation during standby periods. The initialization process may also include calibration verification procedures that compare current system responses with stored reference values to detect potential drift or degradation in component performance.

The user interface subsystem may provide bidirectional communication pathways that enable users to control device operation while receiving feedback about measurement progress and results. The interface coordination may implement state-based operation modes that guide users through sequential measurement procedures while providing appropriate prompts and status information at each stage of the process. The processing circuitry may manage the interface subsystem through dedicated communication channels that carry display data, user input signals, and status information between the central processor and interface components. The interface integration may include priority management systems that ensure critical error messages and safety information receive immediate display attention while routine status updates are presented during appropriate intervals in the measurement sequence.

The power management subsystem may coordinate electrical power distribution throughout the device while monitoring battery status and implementing power conservation strategies that extend operational lifetime between charging cycles. The power coordination may involve dynamic load management that adjusts power consumption based on operational requirements and battery capacity, reducing power to non-essential components during measurement operations to ensure adequate power availability for optical sensing and processing functions. The processing circuitry may communicate with power management circuits through control signals that indicate current operational states and power requirements, enabling the power subsystem to optimize voltage regulation and current distribution based on real-time system demands. The power integration may also include predictive power management algorithms that estimate remaining operational time based on current power consumption patterns and battery status information.

The data flow architecture throughout the integrated system may utilize multiple communication pathways that carry measurement data, control signals, and status information between different subsystems while maintaining data integrity and timing synchronization. The data flow management may implement buffering mechanisms that temporarily store measurement data during processing operations, preventing data loss during periods of high computational activity or communication delays. The processing circuitry may coordinate data flow timing to ensure that measurement data acquisition, signal processing, and result presentation occur in synchronized sequences that maintain measurement accuracy while providing responsive user feedback. The data integration may also include error detection and correction mechanisms that identify and compensate for communication errors or data corruption that could affect measurement reliability or system operation.

The measurement sequence coordination may orchestrate the complex timing relationships between optical sensing activation, data acquisition periods, signal processing operations, and result presentation to provide seamless measurement experiences for users. The sequence management may implement precise timing control that coordinates infrared transmitter activation with photodetector data collection, ensuring that optical measurements occur under stable illumination conditions throughout the acquisition period. The processing circuitry may manage the transition between different measurement phases, including finger positioning confirmation, optical measurement execution, data processing, and result validation, while providing appropriate user feedback at each stage. The sequence coordination may also include adaptive timing mechanisms that adjust measurement duration and data collection parameters based on signal quality assessments and measurement stability criteria.

The error handling integration may coordinate error detection and response mechanisms across all subsystems to provide comprehensive system monitoring and user guidance when measurement problems are encountered. The error coordination may implement hierarchical error classification systems that distinguish between different types of problems, such as optical sensing issues, processing errors, or user interface malfunctions, and provide appropriate corrective guidance for each error category. The processing circuitry may collect error information from various subsystems and correlate error patterns to identify systematic problems that may indicate component degradation or calibration drift. The integrated error handling may also include automatic recovery mechanisms that attempt to resolve certain types of errors through system recalibration or parameter adjustment before requiring user intervention or service attention.

The calibration coordination throughout the integrated system may ensure that all subsystems maintain accurate operational parameters and measurement relationships through systematic calibration verification and adjustment procedures. The calibration integration may involve cross-referencing calibration data between optical sensing components, signal processing algorithms, and display presentation systems to maintain consistency in measurement results and user information. The processing circuitry may coordinate periodic calibration checks that verify system accuracy against stored reference standards or self-calibration procedures that do not require external reference materials. The calibration management may also include adaptive calibration algorithms that automatically adjust system parameters based on measurement history and performance trends to compensate for component aging or environmental variations.

The communication integration between subsystems may utilize standardized protocols and interface specifications that enable reliable data exchange while maintaining compatibility with different component configurations and upgrade possibilities. The communication coordination may implement error checking and acknowledgment mechanisms that verify successful data transmission between subsystems and retry failed communications to maintain system reliability. The processing circuitry may manage communication scheduling to prevent conflicts between different data streams and ensure that time-critical information receives priority access to communication channels. The communication integration may also include diagnostic capabilities that monitor communication performance and identify potential interface problems that could affect system operation or measurement accuracy.

The thermal management coordination may integrate temperature monitoring and heat dissipation strategies across all subsystems to maintain stable operating conditions and prevent thermal-related performance degradation. The thermal integration may involve coordinated activation of heat-generating components such as optical transmitters and processing circuits to minimize peak temperature conditions while maintaining measurement functionality. The processing circuitry may monitor temperature conditions throughout the device and implement thermal protection algorithms that reduce component power consumption or suspend operations when temperature limits are approached. The thermal management may also include predictive thermal modeling that estimates temperature rise based on operational patterns and environmental conditions, enabling proactive thermal management before temperature-related problems occur.

The system diagnostics integration may coordinate comprehensive monitoring of all subsystems to provide early detection of potential problems and enable preventive maintenance before system failures occur. The diagnostic coordination may implement systematic testing procedures that evaluate the performance of optical components, processing circuits, power management systems, and user interface elements during routine operation or dedicated diagnostic cycles. The processing circuitry may collect diagnostic information from various subsystems and analyze performance trends to identify gradual degradation or emerging problems that may not immediately affect measurement accuracy but could lead to future system failures. The integrated diagnostics may also include remote diagnostic capabilities that enable service personnel to assess system condition and performance through communication interfaces without requiring physical access to the device.

The measurement accuracy coordination may integrate various accuracy enhancement mechanisms across all subsystems to provide optimal measurement performance under diverse operating conditions and user populations. The accuracy integration may involve coordinated calibration of optical sensing components, signal processing algorithms, and display presentation systems to maintain consistent measurement relationships throughout the measurement chain. The processing circuitry may implement accuracy verification procedures that compare measurement results with expected values based on system models and historical performance data to detect potential accuracy degradation. The accuracy coordination may also include adaptive accuracy enhancement algorithms that adjust measurement parameters based on signal quality assessments and measurement repeatability criteria to optimize accuracy for specific measurement conditions.

The user experience integration may coordinate all user-facing aspects of system operation to provide intuitive and efficient measurement procedures that accommodate users with varying technical expertise and physical capabilities. The experience coordination may implement progressive user guidance systems that provide appropriate levels of instruction and feedback based on user familiarity with device operation and measurement success rates. The processing circuitry may manage the integration of visual, audible, and tactile feedback mechanisms to provide comprehensive user guidance that accommodates different sensory preferences and environmental conditions. The user experience integration may also include adaptive interface behaviors that modify presentation styles and interaction requirements based on user performance patterns and accessibility needs.

The data security integration may coordinate protection mechanisms across all subsystems that handle patient information or measurement data to ensure compliance with healthcare data protection regulations and maintain patient privacy. The security coordination may implement encryption and authentication protocols that protect data during storage, processing, and transmission operations while maintaining system performance and user convenience. The processing circuitry may manage security key generation and distribution systems that enable secure communication between subsystems and external devices without compromising measurement functionality. The security integration may also include audit logging capabilities that record data access and modification activities for compliance monitoring and forensic analysis purposes.

The maintenance coordination may integrate various maintenance and service functions across all subsystems to provide comprehensive device lifecycle management and ensure continued measurement accuracy throughout the operational lifetime of the device. The maintenance integration may implement predictive maintenance algorithms that analyze system performance trends and usage patterns to recommend optimal maintenance schedules and procedures. The processing circuitry may coordinate maintenance activities such as calibration updates, component testing, and performance verification to minimize disruption to normal device operation while ensuring continued accuracy and reliability. The maintenance coordination may also include remote maintenance capabilities that enable service personnel to perform certain maintenance functions through communication interfaces, reducing the need for device return to service facilities and minimizing downtime for users.

It is to be appreciated that a lesser or more equipped system than the example described above may be preferred for certain implementations. Therefore, any configuration of smart hemoglobin monitoring device may vary from implementation to implementation depending upon numerous factors, such as price constraints, performance requirements, technological improvements, or other circumstances.

A number of implementations have been described. Nevertheless, it will be understood that various modifications may be made without departing from the spirit and scope of the disclosure. Accordingly, other implementations are within the scope of the following claims.

What is claimed is:

1. A non-invasive hemoglobin monitoring device comprising:
processing circuitry coupled to an infrared receiver and a tactile button, the processing circuitry configured to:
detect a finger positioned in a finger placement area based on activation of the tactile button;
acquire multiple sensor readings from the infrared receiver as the finger is positioned;
calculate an average value from the multiple sensor readings; and
determine a hemoglobin concentration based on the average value; and
a display screen coupled to the processing circuitry, the display screen to display the hemoglobin concentration.

2. The device of claim 1, further comprising:
a housing having the finger placement area;
an infrared transmitter positioned within the housing and configured to emit light at a wavelength through the finger placement area;
the infrared receiver positioned within the housing and configured to detect a light transmitted through a finger placed in the finger placement area; and
the tactile button positioned within the finger placement area and configured to be pressed when the finger is properly positioned.

3. The device of claim 1, wherein the processing circuitry is configured to:
acquire the multiple sensor readings from the infrared receiver with an adjustable delay between the multiple sensor readings;
validate the determined hemoglobin concentration by checking whether the concentration falls within a predetermined range in grams per deciliter; and
display an error message on the display when the hemoglobin concentration falls outside the predetermined range.

4. The device of claim 1, wherein the processing circuitry is further to select between a first calculation formula and a second calculation formula based on whether the average value is less than a predetermined value or greater than or equal to the predetermined value.

5. The device of claim 2, wherein the housing further comprises a top enclosure and a bottom enclosure connected by a vertical opening mechanism, wherein the top enclosure houses the infrared transmitter and the display screen, and wherein the bottom enclosure houses a battery and the infrared receiver.

6. The device of claim 1, wherein the processing circuitry is coupled to a memory, the processing circuitry comprises one or more of application processing circuitry or graphics processing circuitry.

7. A method comprising:
detecting, by processing circuitry of a non-invasive hemoglobin monitoring device, a finger positioned in a finger placement area based on activation of a tactile button coupled to the processing circuitry that is further coupled to an infrared receiver;
acquiring multiple sensor readings from the infrared receiver as the finger is positioned;
calculating an average value from the multiple sensor readings; and
determining a hemoglobin concentration based on the average value; and
displaying, by a display screen coupled to the processing circuitry, the hemoglobin concentration.

8. The method of claim 7, wherein the non-invasive hemoglobin monitoring device comprises:
a housing having the finger placement area;
an infrared transmitter positioned within the housing and configured to emit light at a wavelength through the finger placement area;
the infrared receiver positioned within the housing and configured to detect a light transmitted through a finger placed in the finger placement area, and the tactile button positioned within the finger placement area and configured to be pressed when the finger is properly positioned.

9. The method of claim 7, further comprising:
acquiring the multiple sensor readings from the infrared receiver with an adjustable delay between the multiple sensor readings;
validating the determined hemoglobin concentration by checking whether the concentration falls within a predetermined range in grams per deciliter; and
displaying an error message on the display when the hemoglobin concentration falls outside the predetermined range.

10. The method of claim 7, further comprising selecting between a first calculation formula and a second calculation formula based on whether the average value is less than a predetermined value or greater than or equal to the predetermined value.

11. The method of claim 8, wherein the housing further comprises a top enclosure and a bottom enclosure connected by a vertical opening mechanism, wherein the top enclosure houses the infrared transmitter and the display screen, and wherein the bottom enclosure houses a battery and the infrared receiver.

12. The method of claim 7, wherein the processing circuitry is coupled to a memory, the processing circuitry comprises one or more of application processing circuitry or graphics processing circuitry.

13. At least one computer-readable medium having stored thereon instructions which, when executed, cause processing circuitry of a non-invasive hemoglobin monitoring device to perform operations comprising:
detecting a finger positioned in a finger placement area based on activation of a tactile button coupled to the processing circuitry that is further coupled to an infrared receiver;
acquiring multiple sensor readings from the infrared receiver as the finger is positioned;
calculating an average value from the multiple sensor readings; and
determining a hemoglobin concentration based on the average value; and
displaying, by a display screen coupled to the processing circuitry, the hemoglobin concentration.

14. The computer-readable medium of claim 13, wherein the non-invasive hemoglobin monitoring device comprises:
a housing having the finger placement area;
an infrared transmitter positioned within the housing and configured to emit light at a wavelength through the finger placement area;
the infrared receiver positioned within the housing and configured to detect a light transmitted through a finger placed in the finger placement area; and
the tactile button positioned within the finger placement area and configured to be pressed when the finger is properly positioned.

15. The computer-readable medium of claim 13, wherein the operations further comprise:
acquiring the multiple sensor readings from the infrared receiver with an adjustable delay between the multiple sensor readings;
validating the determined hemoglobin concentration by checking whether the concentration falls within a predetermined range in grams per deciliter; and
displaying an error message on the display when the hemoglobin concentration falls outside the predetermined range.

16. The computer-readable medium of claim 13, wherein the operations further comprise selecting between a first calculation formula and a second calculation formula based on whether the average value is less than a predetermined value or greater than or equal to the predetermined value.

17. The computer-readable medium of claim 14, wherein the housing further comprises a top enclosure and a bottom enclosure connected by a vertical opening mechanism, wherein the top enclosure houses the infrared transmitter and the display screen, and wherein the bottom enclosure houses a battery and the infrared receiver.

18. The computer-readable medium of claim 13, wherein the processing circuitry is coupled to a memory, the processing circuitry comprises one or more of application processing circuitry or graphics processing circuitry.
